(19) United States
(12) Patent Application Publication
(10) Pub. No.: US 2024/0198113 A1
(43) Pub. Date: Jun. 20, 2024

(54) WIRELESS DEEP BRAIN STIMULATION DEVICE

(71) Applicant: BIONET SONAR, Newton, MA (US)

(72) Inventors: Jorge Jimenez, Atlanta, GA (US); Emrecan Demirors, Boston, MA (US); Tommaso Melodia, Newton, MA (US); Raffaele Guida, Boston, MA (US); Ryan Theodore Burke, Newton, MA (US)

(21) Appl. No.: 18/556,833

(22) PCT Filed: Apr. 21, 2022
(86) PCT No.: PCT/US2022/025789

§ 371 Date: Oct. 23, 2023

Related U.S. Application Data
(60) Provisional application No. 63/177,428, filed on Apr. 21, 2021.

(51) Int. Cl. A61N 1/372; A61N 1/36

(52) U.S. Cl. CPC A61N 1/37217; A61N 1/36067; A61N 1/36139

(57) ABSTRACT

Systems and methods for wireless deep brain stimulation using ultrasonic waves. Implantable device(s) for intracranial use within a subject may comprise at least one stimulation means, one or more circuits for collecting system data, a receiver, and a transmitter for communications using ultrasonic waves. A wearable controller external to the subject, the wearable controller configured to: communicate with the implantable device(s) using the ultrasonic waves and obtain the system data, analyze the system data to determine whether the subject is experiencing or is expected to experience an adverse medical condition, and communicate with the implantable device(s) using the ultrasonic waves to apply a stimulation to treat the adverse medical condition. The system may be used to treat Parkinson's Disease, for example.

### CROSS REFERENCES TO RELATED APPLICATIONS

This application claims the priority benefit of U.S. Provisional Application No. 63/177,428, filed Apr. 21, 2021, entitled “WIRELESS DEEP BRAIN STIMUATION DEVICE,” which is hereby incorporated by reference in its entirety.

### TECHNICAL FIELD

Embodiments described herein generally relate to the fields of neurostimulation, neuromodulation, and/or deep brain stimulation. Examples uses for systems and methods described herein may include treatment of Parkinson's tremors and epilepsy.

### BACKGROUND OF INVENTION

There is a present and future need to improve the state of the art for wireless networking, monitoring, control, and recharging of medical implant technologies to improve patient outcomes, quality of life (QoL), and/or healthcare economics to make such technologies and treatment more accessible. Improvements in one or more of the following aspects of patient therapy using medical implants may be desirable: (1) reducing primary complications associated with wire tunneling and extracranial leads, (2) reducing the number of system implants and simplifying procedure, (3) improving stimulation through closed-loop Artificial Intelligence (AI) driven modulation, (4) remote high speed monitoring of brain function with ability to tune device function (point-of-care), (5) rapid wireless recharging of system, and (6) reducing costs associated with re-intervention and hospital visits.

There is a need for improved medical implant technologies in Parkinson's Disease. Studies estimate that more than 10 million people worldwide are living with Parkinson's Disease (PD). Approximately 50,000 Americans are diagnosed with PD annually with an estimated 1.2M patients living with PD in the US by the year 2030. Mortality in PD patients increases significantly with age (as associated with dementia), crippling reduction in QoL is the leading characteristic associated with this disease. Cognitive disorders, reduced mobility (bradykinesia, rigidity), and uncontrollable tremors are the main factors that diminish their QoL.

The economic burden of PD in the US exceeds $14.4 billion a year (approximately $22,800 per patient in 2010). The PD population incurs medical expenses more than double that expected for a similarly aged population living without PD. The costliest services are inpatient care ($2.1B), nursing facility care ($1.4B), prescription drugs ($974.8M), hospital outpatient care ($881M), and home care ($776.5M). Indirect costs (e.g., reduced employment) are conservatively estimated at $6.3B (or close to $10,000 per person with PD).

Beyond PD, deep brain stimulation (DBS) systems are finding increasing use in the treatment of Epilepsy patients (3.4M in the US, 30% adults resistant to drug therapy). Treatments for other cognitive diseases and Alzheimer's Disease (preventive) through DBS are currently being studied at preclinical and clinical stages. Brain/Neuro stimulation systems are used or have been developed for many conditions throughout the nervous system, defining a market that is expected to grow above $9.9B by 2026.

PD is a movement disorder characterized by bradykinesia, rigidity, and resting tremor affecting >70% of PD patients, even at an early stage. The benefits of DBS in controlling levodopa-responsive motor symptoms (LRMS) in PD patients and significantly increasing QOL is well-documented. DBS is considered a relatively safe procedure with low mortality: however, it still suffers from complications, including: erosions & infections (5.12%), lead migration (1.60%), fracture or failure of the lead or other implant parts (1.46% and 0.73%, respectively), IPG malfunctions (1.06% of patients), and skin erosions without infections (0.48% of patients), pain at implant site (0.61%), IPG dislocations (0.29%), subcutaneous seroma (0.26%), tethering of extension cable (0.12%), and stricture formation (0.02%). Intervention(s) to correct lead/extension cable complications and battery exchange, regular follow-up and reprogramming visits increases overall healthcare costs and may make the use of such technologies infeasible for many individuals.

In the near future, wirelessly networked systems of implantable and non-implantable medical devices endowed with sensors and actuators will be the basis of many innovative, sometimes revolutionary therapies. However, radio-frequency (RF) electromagnetic waves, which are the physical basis of wireless technologies like Wi-Fi and Bluetooth, have limited penetration depth, low reliability, and high-energy consumption when propagating through biological tissues. Additionally, RF-based technologies are vulnerable to interference from other RF communication systems, and can be easily jammed or eavesdropped. The medical field increasingly relies on sophisticated medical implants, wearables or freestanding equipment. Medical implants are becoming smaller, smarter, and connected. A growing percentage of implants already have data processing and wireless connectivity (for diagnostics, real-time continuous monitoring, or device re-configuration). Wirelessly networked systems of implantable medical devices endowed with sensors and actuators will be the basis of many innovative, sometimes revolutionary therapies. Existing and future applications of wireless technology to medical implantable (as well as wearable) devices will grow into a new market refer to as “The Internet of Medical Things” (IoMT). To enable this, wireless networking devices will need to be based on: (i) miniaturized elements for less invasive deployment: (ii) energy-efficient and reliable data transmission within the body: (iii) minimal power consumption and capabilities to recharge: (iv) secure remote monitoring and control of the implantable device from outside the body: (v) capabilities to process data in real-time: and (vi) re-programmability and coordination of network devices.

A challenge that exists for state of the art medical implant technologies relates to the use of Radio-frequency (RF) electromagnetic waves, and specifically microwaves, which are the physical basis of commercial wireless technologies like Wi-Fi, Bluetooth, and Medical Implant Communication Systems (MICS) are heavily absorbed by biological tissues. As a consequence, (i) RF based transmission heats up tissues, which limits applications in delicate parts of the body such as the brain: (ii) signal absorption limits efficiency, thus requiring larger energy storage/batteries: (iii) tissues also significantly distort and delay RF signals, which causes data transmission to become less reliable: and (iv) absorption limits depth of signal penetration for data or energy transmission.

In contrast, ultrasonic transmission of data and energy does not suffer from the draw backs since mechanical waves are not absorbed to the same extent in biological tissues. Therefore, ultrasonic communication would better enable the creation of implantable Internet of Medical Things (IoMT) communicating devices with and within the human body. The specific communication techniques, hardware, software and protocols described here provide details on how to create an Ultrasonic Wide Band (UsWB) network of medical devices.

Some of the most advanced medical technologies rely on RF based technologies to communicate outside the human body from subcutaneous implants, but still rely on wired/cables connections to communicate to different areas of the body. Cardiac pacemakers and neurostimulation systems are good examples of such technologies where wires/leads are used to send data and energy to other locations in the body from subcutaneous devices. Several complications such as infections, lead failure, pain due to tethering of wires, heart valve malfunction, among others have been associated with the tunneling and chronic implant of such wires and/or leads. Therefore, it would be beneficial to have a wireless network of implantable devices for both intra-corporeal and extracorporeal communication and/or energy transfer to minimize such complications. The UsWB platform allows for intelligent wireless networks for bi-ventricular pacing, deep brain stimulation, and several other applications that may also include remote monitoring capabilities. These networks will reduce mortality/complications in patients with different diseases while reducing healthcare costs associated with in-hospital visits.

### BRIEF DESCRIPTION OF THE DRAWINGS

Having thus described various embodiments of the present invention in general terms, reference will now be made to the accompanying drawings, which are not necessarily drawn to scale, and wherein:

FIG. 1 depicts DBS system design with two implantable leads, one implantable controller, and one external gateway.

FIG. 2 depicts (left) DBS system comprising 2 wireless stimulation leads (WSL), (center) external gateway (EG) for wireless ultrasonic connectivity (>50 kbit/s) and rapid recharge of WSLs and wireless communication with a smartphone application, and (right) wearable controller (WC) enables user to monitor system, receive warning alarms, and measures tremors for closed loop feedback control.

FIG. 3 depicts (left) Ultrasonic communication Packet Error Rate (PER) using UsWB board through different types of tissue at 64 kbit/s, (center) Ultrasonic power transfer through different tissues and depths, where 0.2 mW baseline represents power needed to drive commercially available DBS systems, (right) Discharge time for different capacitor banks at difference communication duty cycles.

FIG. 4 depicts time for 20%, 50%, 80% capacitor (4 F) charge during typical stimulation (3V, 130 Hz, 90 μs).

FIGS. 5A-C depict (FIG. 5A) In vivo testing of 125 kHz link in acute animal per Section A.2.4. up to 35 cms, limited by animal size. (FIG. 5B) photograph of a Bionet watch communicating in humans (wrist to hand at 1.6 m). (FIG. 5C) Frequency plot for prototype dual mode focal transducer (120 kHz, 670 kHz).

FIG. 6 depicts results of testing using 3 different CNN algorithms implemented on FPGA (30 patient Epilepsy database).

FIGS. 7A-E depict various aspects of an implant procedure, according to at least one embodiment of the present disclosure.

FIG. 8 depicts CT showing location of two leads used for bi-lateral stimulation.

FIG. 9 depicts various aspects of WSL electronic circuitry, according to at least one embodiment of the present disclosure.

FIG. 10 depicts a design for dual casing WSL, according to at least one embodiment of the present disclosure.

FIG. 11 depicts various aspects of in vivo protocols for an acute large animal study, according to at least one embodiment of the present disclosure.

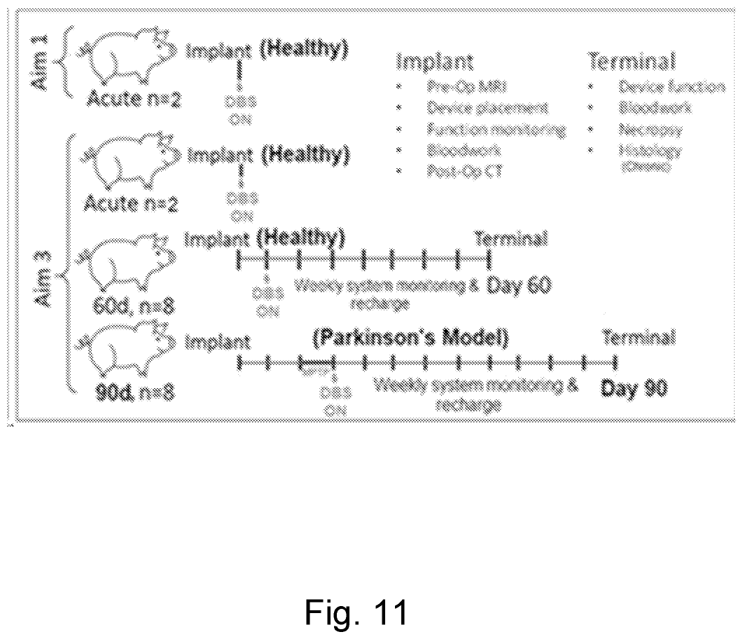

FIG. 12 depicts WSL circuit design using (left) a FPGA and MCU architecture and (right) ASIC unit, according to at least one embodiment of the present disclosure.

FIG. 13 shows the different types of communication links used in the modular networks for both implantable and non-implantable components of the network.

FIG. 14 Block diagram of the principal components of the IoMT platform. Three main block constitute the platform itself: i) Energy Storage unit, ii) Core Units, and iii) Ultrasonic interphase.

FIG. 15 Block diagram of a functional node that can be recharged wirelessly using ultrasonic waves. The addition of a functional unit to the basic IoMT platform structure constitutes a functional node. The energy management unit includes energy harvesting components and energy storage elements. The embodiment described in this diagram also uses a capacitor bank as energy storage for improved safety.

FIG. 16 Diagram of the different nodes/devices that constitute a wireless bi-ventricular pacing network enabled through ultrasonic communication and energy transmission.

FIG. 17 Block diagram of the reprogrammable wireless pacing (RWP) nodes with vascular pressure monitoring. The components of the RWP nodes are similar to those of the generic IoMT platform with the addition of two functional units, a pressure sensor unit and an electric sensor/pacing unit. The RWP nodes are recharged using ultrasonic waves and therefore include the energy management unit with harvesting capabilities.

FIG. 18 Diagram showing different types of treatments using neurostimulation referenced to a specific stimulation site.

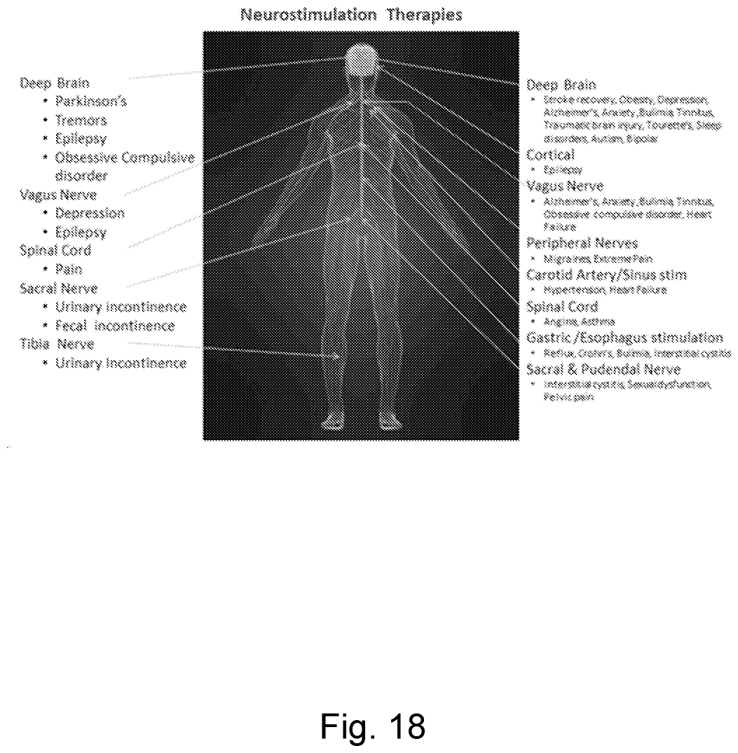

FIG. 19 Diagram of the different nodes/devices that constitute a wireless deep brain stimulation network enabled through ultrasonic communication and energy transmission.

FIG. 20 Block diagram of a reprogrammable stimulation RSN node. The components of the RSN node are similar to those of the generic IoMT platform with the addition of two functional units, an electrical sensor unit and stimulation unit for multi-contact stimulation. The RSN node are recharged using ultrasonic waves and therefore include the energy management unit with harvesting capabilities.

FIG. 21 Diagram of the different nodes/devices that constitute a wireless neuromodulation/CRT network with vascular pressure monitoring capabilities for the treatment of heart failure patients.

FIG. 22A Block diagram of reprogrammable cardiac wireless pacing node and FIG. 22B block diagram of vascular pressure monitoring node, for a wireless neuromodulation/pacing network with vascular pressure monitoring capabilities.

FIG. 23 Diagram of embodiment of deep brain stimulation system wherein RWPs are fully embedded in the brain and controlled by an implantable extracranial controller.

### DETAILED DESCRIPTION

Embodiments of the present invention now will be described more fully hereinafter with reference to the accompanying drawings, in which some, but not all, embodiments of the inventions are shown. Indeed, the invention may be embodied in many different forms and should not be construed as limited to the embodiments set forth herein: rather, these embodiments are provided so that this disclosure will be thorough and complete, and will fully convey the scope of the invention to those skilled in the art. Like reference numbers refer to like elements throughout. The singular forms “a,” “an,” and “the” can refer to plural instances unless the context clearly dictates otherwise or unless explicitly stated.

The invention provides a wireless medical device network comprising: one or more implantable devices for intracranial use within a subject, each of the one or more implantable devices comprising at least one stimulation means, one or more circuits for collecting system data, a receiver, and a transmitter for communications using ultrasonic waves: a wearable controller external to the subject, the wearable controller configured to: communicate with the one or more implantable devices using the ultrasonic waves and obtain the system data: analyze the system data to determine whether the subject is experiencing or is expected to experience an adverse medical condition: and communicate with the one or more implantable devices using the ultrasonic waves to apply a stimulation to treat the adverse medical condition.

The invention provides a wireless medical device network (e.g., as described in combination with features described above and/or below), wherein the adverse medical condition is Parkinson's Disease.

The invention provides a wireless medical device network (e.g., as described in combination with features described above and/or below), wherein the wearable controller comprises an accelerometer/gyroscope (AG) that collects tremor data and is further configured to: determine whether the tremor data is indicative of Parkinson's tremors or normal body function: and responsive to a determination that the tremor data is indicative of Parkinson's tremors, cause the one or more implantable devices to apply a stimulation to the subject.

The invention provides a wireless medical device network (e.g., as described in combination with features described above and/or below), wherein the wearable controller is configured to use a convolutional neural network (CNN) to analyze the system data.

The invention provides a wireless medical device network (e.g., as described in combination with features described above and/or below), wherein the one or more circuits comprises a field-programmable gate array (FPGA) and microcontroller unit (MCU).

The invention provides a wireless medical device network (e.g., as described in combination with features described above and/or below), wherein the one or more circuits comprises an application-specific integrated circuit (ASIC).

The invention provides a wireless medical device network (e.g., as described in combination with features described above and/or below), wherein the one or more circuits are configured to: continuously collect system data: determine, using a first convolutional neural network (CNN) that the system data comprises an abnormal pattern: and in response to the abnormal pattern being detected, initiating transmission of the system data to the wearable controller; and the wearable controller is configured to use a second convolutional neural network (CNN) to determine whether the subject is experiencing or is expected to experience an adverse medical condition.

The invention provides a wireless medical device network (e.g., as described in combination with features described above and/or below), further comprising an external gateway configured to use the ultrasonic waves to provide transcutaneous ultrasonic energy transfer to the one or more implantable devices.

The invention provides a wireless medical device network (e.g., as described in combination with features described above and/or below), wherein each of the one or more implantable devices comprises a dual-mode transducer that operates at a first frequency for signal transmissions with the wearable controller and a second frequency for signal transmissions with the external gateway.

The invention provides a wireless medical device network (e.g., as described in combination with features described above and/or below), wherein the external gateway is further configured to program or re-program the one or more implantable devices via communications over the second frequency.

The invention provides a wireless medical device network (e.g., as described in combination with features described above and/or below), wherein the adverse medical condition comprises an epileptic seizure.

The invention provides a wireless medical device network (e.g., as described in combination with features described above and/or below), wherein the system data is transmitted (e.g., subcutaneously) between the one or more implantable devices and the wearable controller in an unencrypted format.

The invention provides a neurostimulation medical device network comprising: one or more implantable devices for intracranial use within a subject, each of the one or more implantable devices comprising at least one stimulation means, one or more circuits for collecting system data, a receiver, and a transmitter for communications using ultrasonic waves: a wearable controller external to the subject, the wearable controller configured to: communicate with the one or more implantable devices using the ultrasonic waves and obtain the system data: analyze the system data to determine whether the subject is experiencing or is expected to experience an adverse medical condition: and communicate with the one or more implantable devices using the ultrasonic waves to apply a stimulation to treat the adverse medical condition.

The invention provides a neurostimulation medical device network (e.g., as described in combination with features described above and/or below), wherein the adverse medical condition is Parkinson's Disease.

The invention provides a neurostimulation medical device network (e.g., as described in combination with features described above and/or below), wherein the wearable controller comprises an accelerometer/gyroscope (AG) that collects tremor data and is further configured to: determine whether the tremor data is indicative of Parkinson's tremors or normal body function: and responsive to a determination that the tremor data is indicative of Parkinson's tremors, cause the one or more implantable devices to apply a stimulation to the subject.

The invention provides a neurostimulation medical device network (e.g., as described in combination with features described above and/or below), wherein the wearable controller is configured to use a convolutional neural network (CNN) to analyze the system data.

The invention provides a neurostimulation medical device network (e.g., as described in combination with features described above and/or below), wherein the one or more circuits comprises a field-programmable gate array (FPGA) and microcontroller unit (MCU).

The invention provides a neurostimulation medical device network (e.g., as described in combination with features described above and/or below), wherein the one or more circuits comprises an application-specific integrated circuit (ASIC).

The invention provides a neurostimulation medical device network (e.g., as described in combination with features described above and/or below), wherein the one or more circuits are configured to: continuously collect system data: determine, using a first convolutional neural network (CNN) that the system data comprises an abnormal pattern: and in response to the abnormal pattern being detected, initiating transmission of the system data to the wearable controller: and the wearable controller is configured to use a second convolutional neural network (CNN) to determine whether the subject is experiencing or is expected to experience an adverse medical condition.

The invention provides a neurostimulation medical device network (e.g., as described in combination with features described above and/or below), further comprising an external gateway configured to use the ultrasonic waves to provide transcutaneous ultrasonic energy transfer to the one or more implantable devices.

The invention provides a neurostimulation medical device network (e.g., as described in combination with features described above and/or below), wherein each of the one or more implantable devices comprises a dual-mode transducer that operates at a first frequency for signal transmissions with the wearable controller and a second frequency for signal transmissions with the external gateway.

The invention provides a neurostimulation medical device network (e.g., as described in combination with features described above and/or below), wherein the external gateway is further configured to program or re-program the one or more implantable devices via communications over the second frequency.

The invention provides a neurostimulation medical device network (e.g., as described in combination with features described above and/or below), wherein the adverse medical condition comprises an epileptic seizure.

The invention provides a neurostimulation medical device network (e.g., as described in combination with features described above and/or below), wherein the system data is transmitted (e.g., subcutaneously) between the one or more implantable devices and the wearable controller in an unencrypted format.

Various novel deep brain stimulation (DBS) medical systems are described herein that incorporate Ultrasonic Wide Band (UsWB) technology. UsWB technology enables for the first time the creation of wireless intrabody networks of medical implants that can communicated with: and recharge each other. This has not been possible to date because of limitations in penetration (<5 cm) and energy efficiency of even the most advanced radio frequency (RF) based wireless communication and energy transfer technologies (WiFi, Bluetooth, magnetic coupling).

Techniques described herein have been used to demonstrate communication of >1.5 m through the human body and rapid recharging of implanted devices—all while staying within FDA limits for ultrasonic exposure (ultrasound is considered the safest modality of energy transmission and has been used for decades at high intensities, even in the most delicate subject, unborn child).

In one embodiment, a wireless DBS system may be used for the treatment of Parkinson's disease and essential tremor. Such platform will offer the following advantages as compared to current DBS technologies:
Wireless—Eliminates the most frequent complications for DBS, which are directly associated with lead/wire tunnelling, implantation, and long-term failure. Reduction in complications will also decrease healthcare costs associated with re-intervention.
Less Implants—Eliminates the need for an implantable generator, thus also eliminating complications associated with such generator and its implantation procedure.
Simpler Surgical Procedure—Reduced number of implants and no tunnelling of wires will significantly reduce implantation time and the complexity of such procedures.
Artificial Intelligence (AI)-driven closed-loop function—Improves and customizes stimulation to patient needs by means of AI algorithm executed in the implant itself.
Remote monitoring—remote high-speed monitoring of brain function with ability to tune device function (point-of-care) will lead to better, more informed therapy, and reduced healthcare costs associated to in-hospital visits.
Rapid wireless recharging—Improved patient quality of life (QoL) by requiring <20 minutes recharge time every week.

Medical implants are becoming smaller, smarter, and more interconnected with data processing and wireless connectivity. In the near future, wirelessly networks of implantable medical devices will be the basis of many innovative therapies. RF waves, the basis for commercial wireless technologies, are heavily absorbed by tissues (low penetration, energy inefficient) leading to larger implants, and cannot be used in sensitive areas, such as the brain (heating). Ultrasonic communication and energy transmission (mechanical waves) in tissues is a more efficient, safer, and secure alternative. According to at least one embodiment described herein, a wireless network of intelligent implantable devices that can communicate and send energy to each other is implemented. UsWB DBS technology described herein may be utilized for a wide variety of clinical applications, including for Parkinson's Disease (PD), Epilepsy, and other clinical applications (cardiac stimulation, neuromodulation, and prosthetics).

Although DBS is generally considered a safe procedure, the most frequent complications are directly associated with lead/wire tunneling, implantation and long term failure, therefore wireless is a word often used when describing the future of DBS and neuro-stimulation (NS) therapies. Commercially available DBS and NS systems use Bluetooth technologies to communicate externally, to monitor or reprogram the system. Some additionally use magnetic induction to recharge. However, these devices have wired leads, and provide limited remote monitoring capabilities. Although closed loop DBS systems have been proposed and under development for many years, their advancement has been limited by hardware (size/efficiency) required to process big data. Only one FDA approved DBS system has successfully implemented closed loop control, but it is a reactive system not a predictive system in absence of AI.

Advances in DBS system are described in this disclosure. As described in greater detail below; DBS systems implemented in accordance with this disclosure will be the first medical technology to implement a highly innovative intra-body wireless network. In at least one embodiment, a DBS system described herein is designed to i) eliminate/reduce complications and re-interventions associated with wire/lead tunneling, lead failure, lead infection or migration and reduce healthcare cost due to in hospital visits by allowing for remote monitoring and re-programming: further wireless implies not wires through skull, neck or chest which will allow for more leads to be used in more advanced/complex DBS therapies, ii) eliminate the need for an implantable generator, reducing the number of procedures and surgical time, iii) enable high data rate upload of system and brain function into the cloud for better informed clinical decisions, iv) allow for closed loop feedback, and v) use embedded AI to improve stimulation function and PD therapy efficacy. In terms of advancement of the overall medical technology, UsWB communication and energy transfer technology described herein will enable for the first time communication through a high speed intra body network, thus enabling paradigm shifting multi-organ and multisystemic treatments connecting different implants. Further, energy efficient AI that can be implemented directly on the medical device will allow for more patient specific treatments, through active monitoring and learning from patient body response. These new tailored treatments can apply predictive/preventive functions to improve patient outcomes and QoL. High speed connectivity will also allow for informed remote patient monitoring to make well based on large amounts of data in real time. Concern over hacking or interference of medical devices will also be addressed, since at least intrabody network (close loop function) can't be hacked remotely as direct physical contract with the patient is required. Finally, high sampling rate monitoring of brain waves in real-time will also advance basic understanding of brain function with patients outside of a clinical or research setting.

Additionally, a continuous monitoring data stream of more precise information may be provided to test the broad parameter space possible with deep brain stimulation (DBS), which to date has been significantly based on trial-and-error without continuous and real-time collection of data. Network data streaming will also provide massive amounts of sensor-measured data, enabling use of AI for paradigm-shifting patient care and clinical data management. A generalizable platform that enables brain monitoring, closed-loop response, and embedded AI, will also allow the system to be adaptable for other applications (e.g., Epilepsy) which can, in at least some embodiments, be achieved through software upgrades that can be implemented without requiring additional invasive procedures. Various embodiments described herein utilize the innovative UsWB technology, since traditional wireless communication and recharging using radio frequency (RF) waves is limited by physics: RF waves are heavily absorbed by tissues.

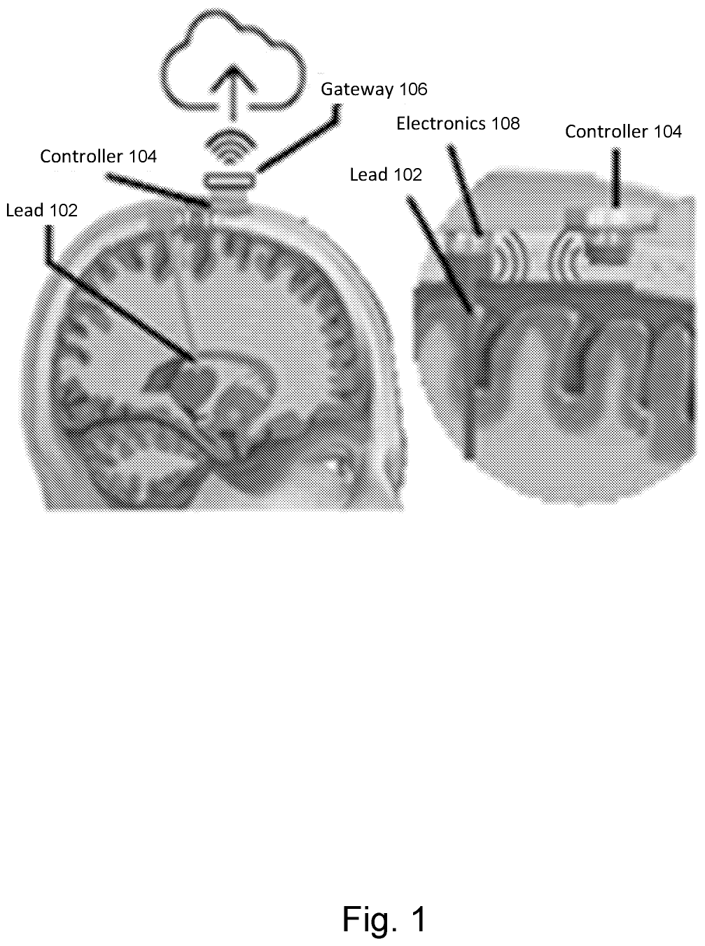

FIG. 1 illustrates the rigors of prior research (Phase I study). FIG. 1 generally depicts a DBS system design proposed in Phase I with two implantable leads 102, one implantable controller 104, and one external gateway 106. A DBS system with the Phase I design includes: i) Two skull embedded wireless stimulation leads (e.g., lead 102), ii) Implantable controller 104 placed in head subcutaneously, and iii) external recharging/reprograming gateway 106 (e.g. for delivering internet connectivity). Electronics 108 may be in communication with implantable controller 104.

This design can be modified with the following additional features: reducing the number of implantable components by removing the implantable controller and replacing it with a wearable. Having fewer implants will reduce the number and complexity of surgical procedures required for deployment (Currently 3 implants, 2-3 procedures). In contrast, embodiments described herein may take an approach in which two implants (pair of leads) are to be deployed in a single surgical intervention.

Additionally, any component embedded within the brain may be designed with a tether to the skull to allow for easy repositioning or removal. Currently, almost all wire/lead complications occur in the portion of implants between the skull, neck and chest. Since lead complications/failures intracranially (stimulation site to skull) are negligible, placing a physical section of lead or tether intracranially doesn't remove any value from system but does reduce risk.

Any of the features described above may be defined as clinical requirements above to the original DBS system design of FIG. 1 to develop an improved UsWB DBS system that was designed, manufactured, tested, and successfully demonstrated feasibility.

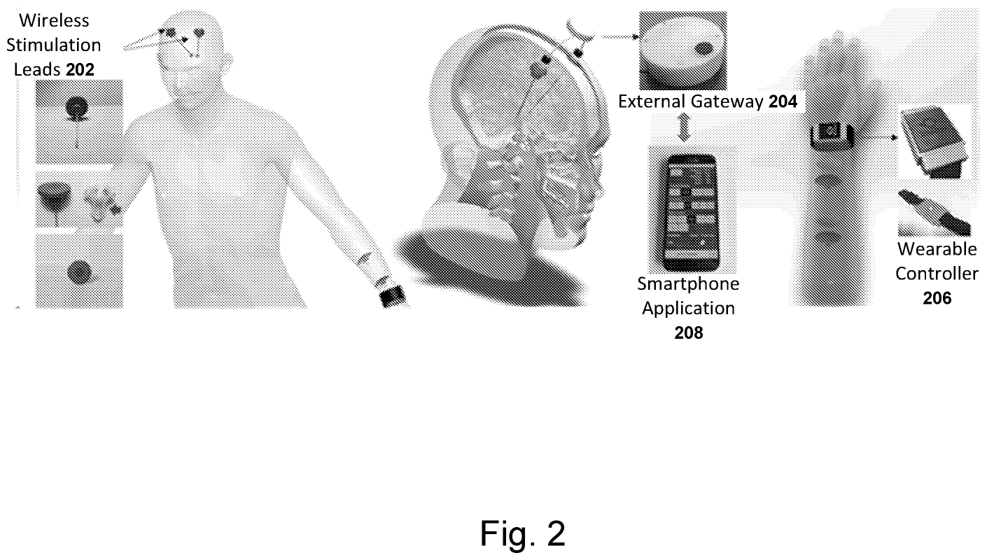

FIG. 2 depicts a DBS system, according to at least one embodiment of the present disclosure. In FIG. 2, a DBS system comprises two wireless stimulation leads (WSL) 202, external gateway (EG) 204, and wearable controller (WC) 206. WSL can be implanted in a single procedure through small incisions in the skull. The incisions may be 14 mm in size, according to at least one embodiment. However, in other cases, the incisions may be smaller or larger. In the right-portion of FIG. 2, a wearable controller is depicted. A wearable controller may be in the form of a watch or smart watch, arm band, or bracelet. Wearable controller may be utilized by a user to monitor systems, receive warning, alarms, and may be used to measure tremors for closed loop feedback control. In the center-portion of FIG. 2, an external gateway is depicted for providing wireless ultrasonic connectivity and/or rapid charging. The external gateway may be used for two-way communications with a smartphone application 208. Connectivity may be provided at a rates exceeding 50 kbits/sec, for example. Rapid charging provide for a full charge of WSL on the order of 10-20 minutes. In various embodiments, a DBS system implemented in accordance with FIG. 2 provides real-time remote monitoring of patients for effective point-of-care therapy. Photographs of prototype leads are depicted in FIG. 2, and have been tested in vitro and in vivo.

In accordance with at least one embodiment, a DBS system—such as depicted in FIG. 2—uses a radically new UsWB technology to deliver bi-lateral stimulation for the treatment of PD patients. An implementation of a DBS system as described herein comprises three components: a plurality of implants (wireless stimulation leads), (2) a wearable controller (WC, watch, bracelet or arm band), and (3) a temporary recharging and communication external gateway (EG). In some embodiments, a DBS system comprises a single stimulation element or implant that may be used to conduct unilateral stimulation.

A DBS system described herein may comprise a single or a plurality of wireless stimulation leads (WSL), for example, exactly two WSLs according to at least one embodiment. In some embodiments, a single WSL may be used. Wireless stimulation leads may be directional leads, designed with a geometry that allows for the electronics to be embedded within the standard cranial opening, for example, 14 mm Diameter×1 cm, defined for PD DBS procedures. In at least one embodiment, a flexible shaft extends from the electronics to the stimulation site and is adjustable in length, 1.5 mm diameter, and has a plurality of electrodes located at its tip. In an exemplary embodiment, 8 Platinum/Iridium electrodes are located at the tip. A curved ultrasonic transducer may be used for rapid recharging. In some embodiments, the curved ultrasonic transducer sits above the cranial surface (3 mm height) to allow for rapid recharging and (i) high-speed bidirectional data transfer (>50 kbit/s) to the EG, (ii) communication between the two WSLs for synchronization, and (iii) long-range secure ultrasonic communication with WC. WSL energy storage capacitors allow for 6 to 7 days of function without recharge (>10 Year capacitor life). A wireless recharge to full battery or near full (e.g., 99%) using transcutaneous ultrasonic energy may be achieved in under 20 minutes.

A DBS system described herein may comprise a wearable controller (WC) in the form of a watch or arm band or any other wearable form factor in contact with any other section of the body, and uses an ultrasonic intra-body bi-directional communication scheme to connect with the WSLs to monitor system function and provide user alarms. For example, a first ultrasonic signal centered at 125 KHz (acoustic signal range may include a range of 10 KHz-5 MHZ) may be used for communications between the WSLs and WC. It is noted that the wearable may be a device that is external to—that is, not implanted in—a subject and may be attachable to the subject, for example, via a wristband or other attachment means. A wearable controller may have integrated sensors such as accelerometer/gyroscope (AG) sensors to monitor arm movement or tremors. Data from AG may processed by a complex AI algorithm implemented using hardware and/or software to discern between normal motion and tremors. For example, the WC may be a wearable hardware device that comprises specialized hardware in the form of in a field-programmable gate array (FPGA) with circuitry designed with an AI model specifically for detecting PD tremors or other conditions. Tremor data may be processed and combined with system data in a closed loop to optimize stimulation within prescribed ranges, as programmed by the PD patient's physician. Adjustments outside the closed-loop function range will only be done through the EG with physician input remotely or in situ. The security of DBS systems described herein may be more secure and less vulnerable to hacking and other electronic attacks, as closed loop communication is virtually un-hackable as ultrasonic waves travel only within the patient's body, alleviating medical device hacking concerns. For example, signals that travel “over the air” may need security measures such as encryption, but ultrasonic waves that travel exclusively within a patient's body may be transmitted without the additional overhead of encryption. This can result in beneficial improvements to the implantable systems, such as obviating the need to store cryptographic material—which oftentimes requires specialized hardware—and reducing the computational and power needs for transmission. These benefits are especially important in the context of the WSL implants, as these improvements can reduce the frequency of charging and increase the lifespan of the WSL, reducing the frequency at which users may be subject to invasive procedures. In some embodiments, signals may be encrypted or encoded, adding additional security features even with using UsWB. In some embodiments, encryption is not needed to protect communications, as ultrasonic waves travel within the patient's body and may be practically impossible or infeasible to be intercepted.

For epilepsy, deep learning has offered the possibility of predicting seizures before they happen, this involves classifying the distinction between the pre-seizure period (preictal) and non-seizure period (inter-ictal), usually using electroencephalography (EEG) or intracranial electroencephalography (iEEG) data as input to the deep learning network (DLN). In terms of response time and clinical applicability, systems with in situ DLN are required so that a closed loop response is implemented. Therefore, DLN need to be optimized in order to fit into miniaturized hardware which may fit in form factors amiable to implants or wearables. Miniaturized programmable hardware include FPGAs (Field Programmable Gate Arrays) and MCUs (microcontrollers) that can be used to hold artificial intelligence (AI) algorithms to process brain signals in situ in order to predict a seizure and provide appropriate stimulation in a closed loop routine. The FPGA contains modules for the DLN and physical communication layer. The iEEG data is sent directly to the FPGA which performs forward propagation through the DLN to get an input classification. The classification is then processed in the MCU (either at the implant or external node) with other classifications for a majority vote amongst a multitude of spatially and/or temporally diverse DL classifications to increase accuracy and mitigate false classifications. Once an agreed upon classification is made, the MCU sends the classification back to the FPGA for coordination of the closed loop stimulation protocol.

Different deep learning approaches may be used for seizure classification. Convolutional neural net-works (CNNs) can be used and have shown good results in seizure prediction. They are fairly simple to implement into hardware and serve as the basis for many more sophisticated deep learning techniques. Such techniques include Stacked Convolutional Autoencoders. This involves an encoding CNN attached to a decoding CNN where the input and output layer are the same size and shape. In addition, Long Short-Term Memory (LSTM) networks are a form of recurrent neural networks and have shown promise in sequence prediction and translation especially in seizure prediction. LSTMs offer, as the name suggests, a memory in the network which can prove useful if pre-seizure states act similarly for specific patients. For the embodiments of the deep brain stimulation system the hardware that runs the DL algorithms may be located on a or multiple wearables, on one or multiple implants, or partially held in one or multiple implants or one and multiple wearables.

A DBS system described herein may comprise an external gateway (EG). An external gateway as described herein may refer to a portable device. The dimensions of an EG may, for example, be in the range of 5 cm diameter, 2 cm height. The EG uses ultrasonic transcutaneous link (700 KHz, range 10 KHz to 5 MHz) for bi-directional communication with, and recharging of, WSLs and to reprogram, recharge and receive real-time brain monitoring data from the WSL. The EG contains Bluetooth connectivity or other short-range wireless communications means to reprogram or download data from the WSL through a phone or computer-based application. The EG battery may provide for 12 hours of continuous communication and 3 complete system recharges when un-tethered.

A phone or computer-based application 208 may be utilized to communicate with the system and can reprogram all stimulation specifications and assess system function. Any other wireless element beyond such phone may be also used to connect the intra body network with the internet, allowing for remote control or monitoring. In some embodiments some of the elements of the intrabody network may communicate with a network outside the body using radio frequency based signals. The external application may use wireless means, such as a Bluetooth link, to control the EG. The application can send data to the cloud as well as provide remote reprograming or monitoring. This application should also enable real-time brain function streaming.

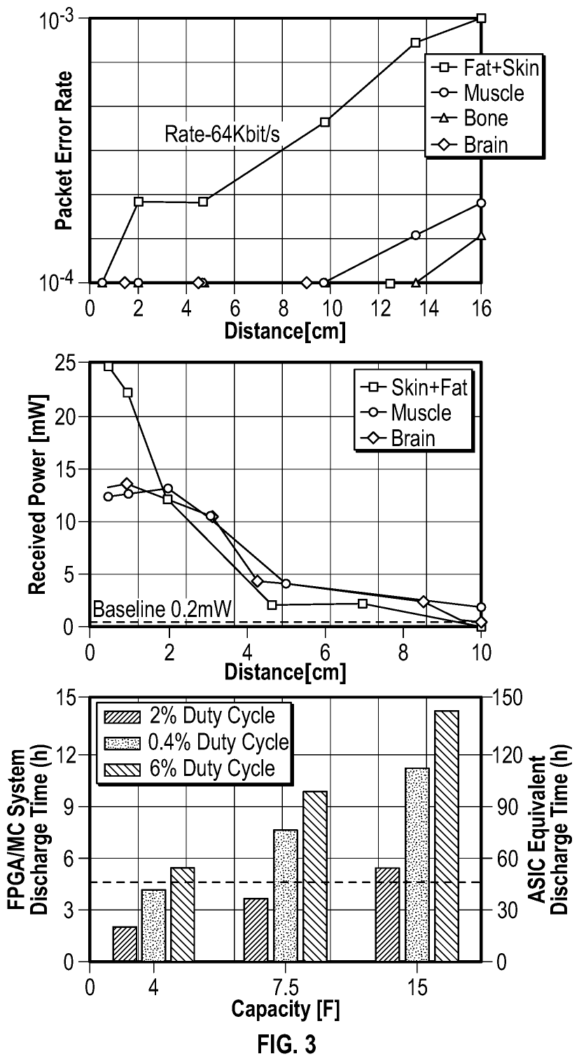

FIG. 3 depicts operating characteristics of various forms of wireless communications. In the left-portion of FIG. 3, communication packet error rate (PER) using UsWB is plotted against distance through different types of tissue at a rate of 64 Kbits/sec. In the center-portion of FIG. 3, ultrasonic power transfer through different tissues and depths is plotted against different types of tissue, where 0.2 mW baseline represents power needed to drive commercially available DBS systems. In the right-portion of FIG. 3, discharge time for different capacitor banks at difference communication duty cycles. In various embodiments, the system uses an ASIC chip rather than FPGA/MC architecture (as proposed in Phase I). Additionally, the ASIC equivalence was calculated as the dashed redline represents 48 hours of independent function.

In some embodiments communication between the EG and WSL may be conducted using Bluetooth, Wifi, or other forms of radio frequency based communication, including but not limited to: WiFi, Bluetooth, ZigBee, Z-Wave, 6LoWPAN, Thread, WiFi-ah (HaLow), 2G (GSM), 3G & 4G, LTE Cat 0, 1, & 3, LTE-M1, NB-IOT, 5G, NFC, RFID, SigFox, LoRaWAN, Ingenu, Weightless-N, Weightless-P, Weightless-W, ANT & ANT+, DigiMesh, MiWi, EnOcean, Dash7, WirelessHART, WirelessHART is built on the HART.

In some embodiments communication between the WC and WSL may be conducted using Bluetooth, Wifi, or other forms of radio frequency based communication, including but not limited to: WiFi, Bluetooth, ZigBee, Z-Wave, 6LoWPAN, Thread, WiFi-ah (HaLow), 2G (GSM), 3G & 4G, LTE Cat 0, 1, & 3, LTE-M1, NB-IOT, 5G, NFC, RFID, SigFox, LoRaWAN, Ingenu, Weightless-N, Weightless-P, Weightless-W, ANT & ANT+, DigiMesh, MiWi, EnOcean, Dash7, WirelessHART, WirelessHART is built on the HART.

In some embodiments the implantable elements of the system can be recharged wirelessly using, but not limited to: magnetic induction, magnetic coupling, tesla coils, electric field coupling, radio reception, heat transfer, mechanic motion charging, radiation, capacitive coupling, light coupling, galvanic coupling, wireless power transfer, and other form of RF based wireless energy transmission.

Systems described herein may have one or more of the following target metrics:

TABLE 1
Summary of Components Level Testing of DBS System

| Functionality | Target Metric | Result | |
|---|---|---|---|
| UsWB | Reliable <0.1% PER | Reliable 0.01-0.1% PER | Exceeded |
| Communication | Data rate >50 Kbit/s | Data rate 64 Kbit/s | (FIG. 3, left) |
| | Distance >15 cm | Distance >16 cm | |
| | Power 50% more efficient | >10 cm no reliable RF | |
| | RF at 15 cm | communications | |
| Ultrasonic | Transmit enough energy | 1.5-2 mW at 8 cm | Met |
| Recharging | (0.2 mW) to maintain | 0.2-1.5 mW at 10 cm | (FIG. 3, center) |
| | function at 8-10 cm depth | | |
| Stimulation Range/ | Frequency 0-250 Hz | Frequency 0-250 Hz | Exceeded |
| Accuracy | Amplitude 1-10 V | Amplitude 1-24 V | |
| | Pulse width 30-90 μs | Pulse width 30-200 μs | |
| | Accuracy <10% error | Accuracy <1% error | |
| | | (36 Mhz crystal | |
| | | generator) | |
| Energy Storage | Equivalent energy storage | With duty cycles of | Exceeded |
| | for 24-48 hr ASIC | 0.4% 37 hr (4F), 75 hr | (FIG. 3, right) |
| | equivalent capacity | (7.5Fr), 97 hr (15Fr) | |

In vitro study of component level testing is described in greater detail below.

As part of components level testing, overall capacity of the individual technology/components was assessed for all boards and components designed to be part of the fully assembled DBS system. Ultrasonic Communication (speed, reliability, package error rate (PER)), Energy Transfer (time to recharge capacitors), Stimulation range (0-250 Hz, 1-10V, 30-200 μs), and Energy Storage (recharge free work cycle) functions were evaluated independently. Communication and energy transfer were tested at different tissue depths using different clinically relevant tissue samples. All design criteria and performance specifications were met or exceeded (Table 1, FIG. 3). An ASIC equivalency of 10× was used for comparing FPGA/MC based circuits to an ASIC based system life cycle (FIG. 3, right). The 10× published equivalency also correlates well to the data showing that the stimulation function with MC uses approximately 2 mW, whereas custom DBS systems using ASIC consume approximately 0.2 mW under typical settings. Additionally, a UsWB intrabody communication scheme (125 Khz) for the WSL to WC link was demonstrated at distances >1 m, as presented in detail below.

As part of the in vitro study, system level testing was conducted using fully assembled devices and software application: performing integrated (communication, stimulation, recharging) functions at clinically relevant tissue depths for the DBS System, including: i) Transcutaneous (at scalp, skin-fat-muscle) energy transfer and communication between EG and WSL, and ii) Communication along (parallel to) skin surface (skin+subcutaneous) between WSL and WC.

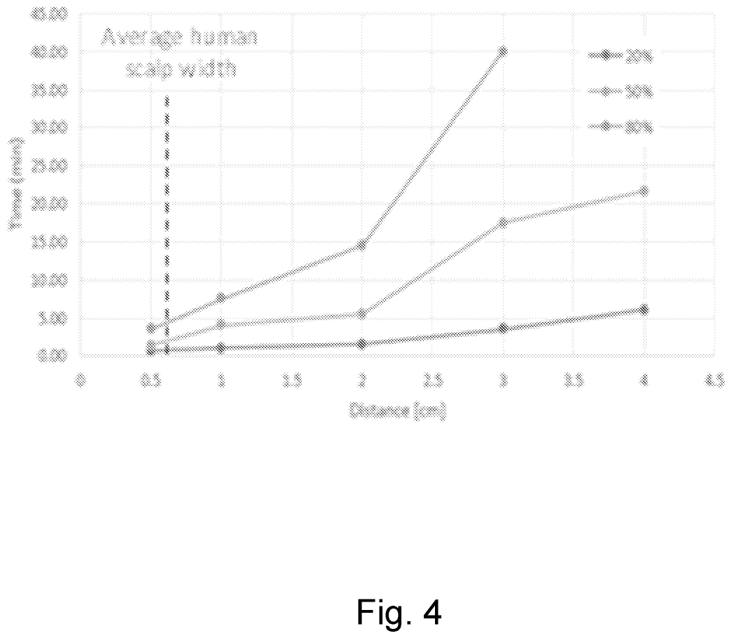

Energy transfer was examined as part of the system level testing. Charging times to 80% capacitor charge (4F, 5 min) through average scalp thicknesses (2-6 mm) were demonstrated in FIG. 4. Charging times were linear with increasing capacity (9 min @ 7.5 F, 19 min@15 F for 80% capacity). These data demonstrate that a 15 F capacitor can provide up to 142 hours (6 days, FIG. 3) energy between charges and rapid (<20 minutes) transcutaneous recharging. FIG. 4 illustrate a graph of time for various levels of capacitor charge during typical stimulation, plotted against distance. The average human scalp width is depicted as a vertical, dashed redline.

Communication between EG and WP was examined as part of the system level testing. Assembled devices communicated through the scalp with the same performance as individual Components Level Testing (FIG. 3), and exceeded requirements as evidenced by ability to communicate at 64 kbit/s with a PER of 0.05%. (no meaningful throughput degradation).

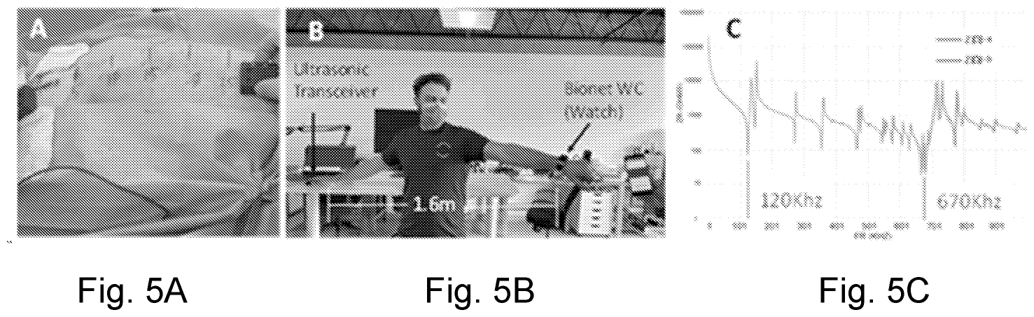

Communication between WSL and WC was examined as part of the system level testing. Long range (FIGS. 5A-5B) body surface communication between a wearable and wireless DBS system (beyond scope of Phase I study) was also developed to meet clinician requirements (e.g., as discussed above). This WC prototype uses 125 kHz piezo and bi-directional hardware packaged into a watch form factor. Testing of this prototype demonstrated reliable (PER<0.1%) body surface communication at distances >1.5 m at 10 kbit/s in vitro, in vivo, and in humans. Human factor testing by several members of our team FIG. 5B demonstrate ability to communicate from the Bionet watch, the WC, to a receiver on the hand of the other arm (>1.5 m Distance).

In order to use a single piezo per WSL and have the capability to use ultrasonic signals centered at 700 kHz and 125 kHz for transmissions with EG and WC, respectively, we developed and fabricated a dual-band concave/convex focal transducer. The first prototype operated at 120 Khz (Diameter mode) and 670 Khz (Thickness mode) and as shown in FIG. 5C. A second prototype (150 Khz, 720 KHz) with a form factor that meets WSL design criteria may also be utilized, if available. Several piezo for factors including, discs, cylinders, concave convex bowls, spheres plates, membranes, hemispheres, among others may be used to produce several acoustics signals at different frequencies by the dame transducer in order to provide better efficiency in accounting data transfer or energy transfer.

Aspects of independent function for implants (WSL) are described herein. Two different communication duty cycles (WSL to WC) were tested during continuous stimulation with medium (0.4% duty cycle) and high (0% duty cycle) efficiency schemes. The 0.4% duty cycle implies communication between WSL and WC every minute. In the high efficiency duty cycle the WSL communicates with the WC when there is a system alarm or when the WC sends a signal to request a system update. During normal operation of the system, alarms should rarely occur, and user would seldom ask for a stimulation update. WSL Capacitor discharge time was measured for both cycles under typical stimulation settings (3V, 130 Hz, 90 μs). System performance was similar to component level (FIG. 3) as evidenced by achieving 48 hour independent function (ASIC equivalent) with 4 F and 7.5 F WSLs using the high efficiency scheme, and with 7.5 F using medium efficiency scheme. In some embodiments, 15 F capacitor bank may be used, space permitting. Therefore, system requirements were met by the preferred configuration.

Aspects of smartphone applications for controlling DBS systems are described herein. A smartphone application (e.g., running in a smartphone as depicted in FIG. 2) allowed transcutaneous UsWB reprogramming of the WSL through the EG, and ability to vary the amplitude (0-10V), frequency (0-250 Hz) and pulse length (30-200 μs) of the stimulation system. This application was successfully used to control and monitor DBS systems during system integration in vitro and acute animal testing, using Bluetooth from phone to EG, and then through transcutaneous UsWB from EG to WSL.

Aspects of AI and closed loop control are described herein. Miniature AG sensor was integrated into the electronic board used in the WC, which allows for measurement of tremor intensity and frequency. These data may be used to train artificial intelligence algorithms (Regression Algorithms, Instance-Based Algorithms, Decision Tree Algorithms, Clustering Algorithms, Association Rule Learning Algorithms, Artificial Neural Network Algorithm, Deep Learning Algorithms, supervised, semi-supervised, unsupervised, reinforcement) including but not limited to convolutional neural networks (CNN) implemented on the WC to discern Parkinson's tremors from normal body function. AG Data, WC CNN processing hardware, and bi-directional communication between WSL to WC may be the basis for closed loop feedback modulation of stimulation. To date, most AI solutions rely on cloud or edge systems: however, in at least one embodiment, a DBS system described herein is able to implement CNNs in miniaturized embedded hardware using flexible libraries that enable implementation of complex CNNs in low-power FPGAs (15× less energy use than and 17× less latency CPUs). For example, an Xylinx Zynq-7000 System on Chip (Dual-core ARM and FPGA, 1 cm×1 cm) or other suitable SOC with form factor that easily fits within the WC form factor may be utilized.

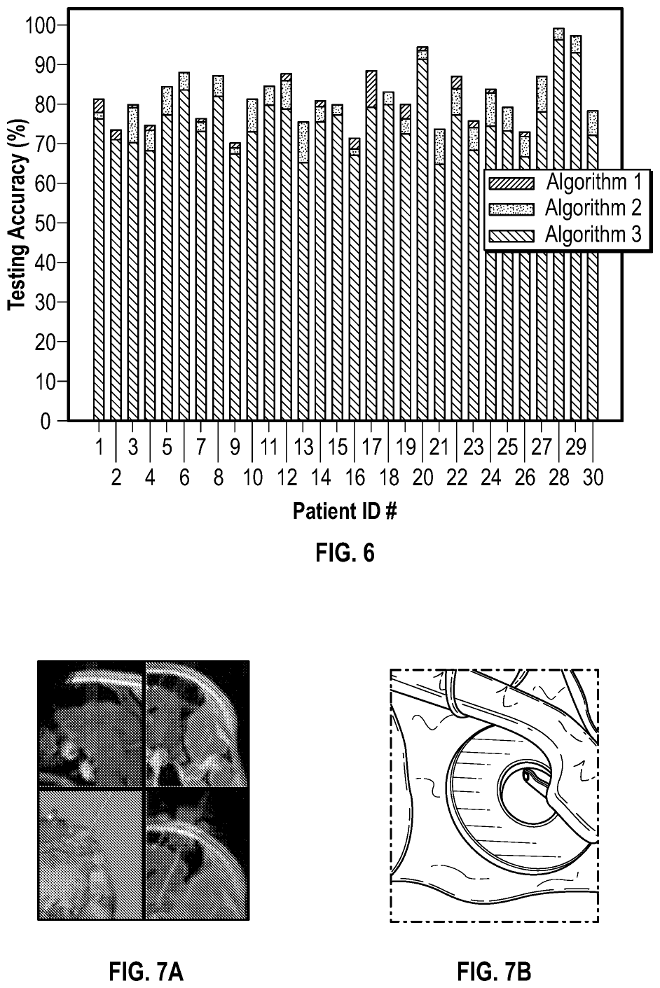

In various embodiments, the embedded CNN is trained and tested for Epileptic seizure prediction. In various embodiments, the CNN or other suitable AI-based model uses a database to generate predictions of whether an Epileptic seizure will occur. A CNN as described herein may accurately predict the onset of an Epileptic seizure in 82% of the cases at least one hour before onset (FIG. 6). The next step is to test the CNN with a control group of patients, which were not part of the data set used for initial training. In various embodiments, the CNN may be trained to use measurements of tremor intensity and frequency to discern Parkinson's tremors from normal body function.

FIG. 6 depicts results of testing using 3 different CNN algorithms implemented on FPGA on a patient Epilepsy database, according to at least one embodiment of the present disclosure. Two of the tree CNN predicted with 70% accuracy a seizure 1 hour in advance of actual event for all patients. The best performing CNN predicted the event 1 hour in advance with an accuracy of 82%±7%.

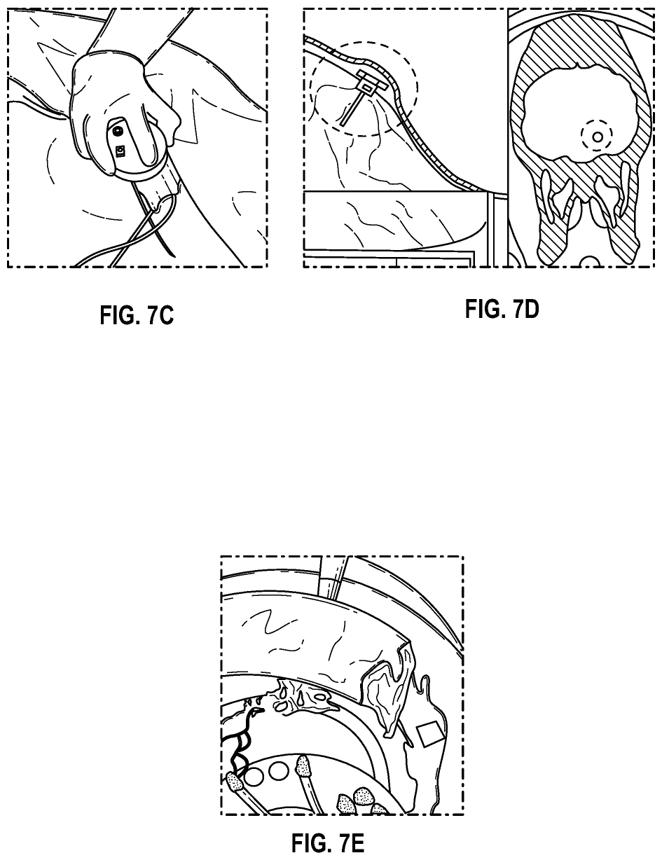

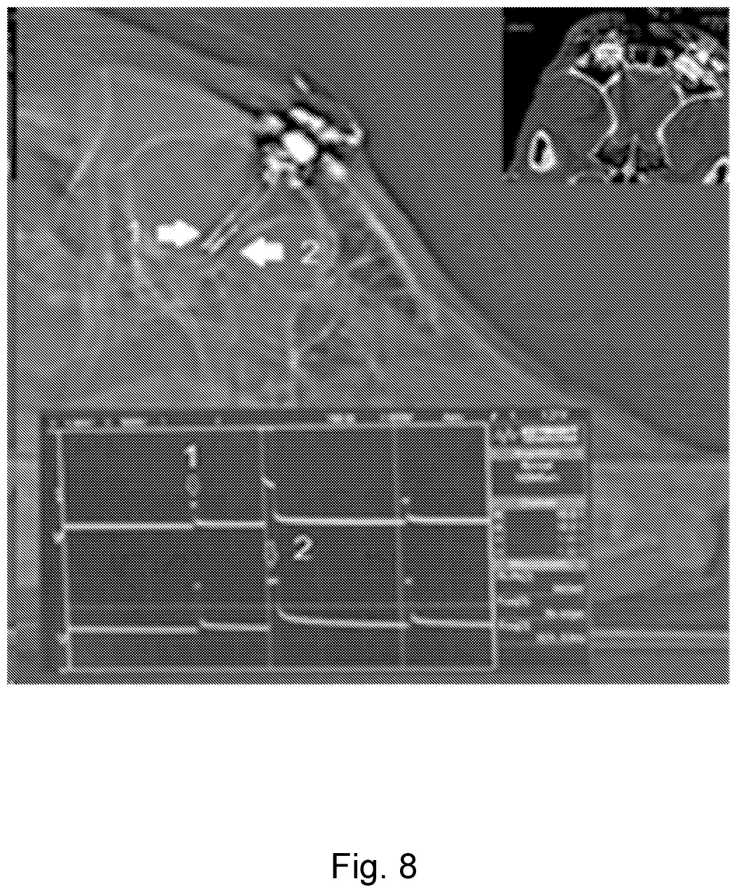

Aspects of acute in vivo study are described herein. A DBS system as described herein was implanted in a large animal model following clinical protocol (pre-op MRI, neurosurgery, post-op CT), tested over a wide range of stimulation settings (mild, typical, aggressive), and was evaluated for ability to recharge system using UsWB technology, as depicted in FIG. 7A-FIG. 7E. The full stimulation, reprogramming and recharging protocol was completed in all subjects. The ability to change stimulation settings using the smartphone application, through the EG and transcutaneous into the WSL for mild, typical and aggressive stimulation setting, was also demonstrated. Amplitude (1-10V), frequency (50-250 Hz), and pulse width (60-200 μs) were varied, and precision of pulse settings was verified using the oscilloscope (<5% error). Continuous WSL function was observed for the duration of the study. When recharging the system to 80% capacity, recharge time was <9 min and tissue or transducer showed no signs of heating. A second WSL was then placed in the second animal demonstrating independent bi-polar stimulation, as seen in FIG. 8.

FIG. 7A-FIG. 7E are described herein. FIG. 7A depicts a pre-Op MRI that was used for surgical planning/navigation. FIG. 7B depicts WSLs that were implanted using medical grade 14 mm drill bit. Flange height can be reduced (3 mm) in response to aesthetics concerns raised with current profile. FIG. 7C depicts External Controller (EC) that was used to reprogram stimulation settings by sending control signal wirelessly using UsWB. EC was also used to recharge the system at the end of the procedure to quantify the recharge rate. Wires exiting the implant were used to: i) monitor the voltage and current of the capacitors to quantify recharge rate, and ii) connect oscilloscope to WSL monitoring electrodes (electrodes (1, 4) stimulate: (2, 3) monitor) to independently monitor stimulation signals in high resolution. FIG. 7D depicts post-Op CT showed lead was correctly placed on target for both animals. FIG. 7E depicts photograph of blood ingression into first prototype device observed during first implant procedure, which was later successfully replaced with second prototype device in same animal.

FIG. 8 depicts a CT showing location of two leads used for bi-lateral stimulation. Oscilloscope signal shows each lead working independently as per programming through EG.

A DBS system was implemented and successfully demonstrated feasibility as evidenced by intrabody reliable ultrasonic communication through different tissues at high data rates (10-64 kbit/s) at penetration up to 16 cm using 700 kHz and >1.5 m at 125 kHz, and energy transmission up to 10 cm depth, according to at least one embodiment. These metrics are within FDA limits for tissue exposure to ultrasonic waves. A DBS system implemented herein demonstrated full functional performance metrics in both in vitro and in vivo models at clinically relevant implant depths and with rapid recharging (<10 minutes). The ability to achieve independent stimulation duration of >48 Hrs (4 F energy storage) was also demonstrated, and >6 days (15 F).

In various embodiments, a DBS system in accordance with the present disclosure implements a closed loop system using a hardware and software platform.

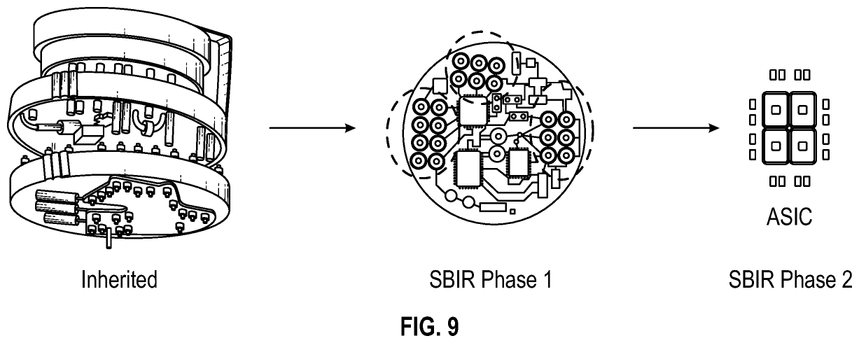

Advanced electronic element packaging techniques & software (System-on-Chip, ARM D-5 Development Studio) were used to package dual board UsWB electronics into a single substrate (FIG. 9): (B) Uncoated dual frequency (120 KHz, 670 KHz) transducer was manufactured: (C) WSL stimulation with FPGA/MC architecture doesn't allow ability to achieve lower energy levels: (D) WSL mechanical design did not include variable lead length to reach stimulation site and used only 4 electrodes to stimulate (clinical practice moving to 8 electrode directional leads, although 2 or more electrodes are required): (D) EG hardware meets requirements for size, recharging and communication speed, Bluetooth connectivity, and energy capacity, but needs redundancy for added safety and durability and a second ultrasonic transducer and corresponding interface to recharge and communicate both WPLs in parallel (bi-lateral DBS): (E) WC form factor (watch) can communicate bi-directionally at >10 Kbit/s over long ranges (>1.5 m) through the surface of the body and has integrated accelerometer and gyroscope sensors, but requires dual band piezo/connectivity to communicate with WSL: and (F) smartphone application—need to improve interface based on human factor testing, software architecture, and redundancy to meet medical device software specifications: (G) Both 10 Kbit/s and 50 kbit/s communications schemes allow for real-time sampling and data transfer of brain functions at rate significantly above those used by iEEG (256 Hz). Further studies may be focused on determining optimal brain function sampling rate >256 Hz that would work with 10 Kbit/s closed loop communication, with acceptable buffer memory and energy consumption requirements.

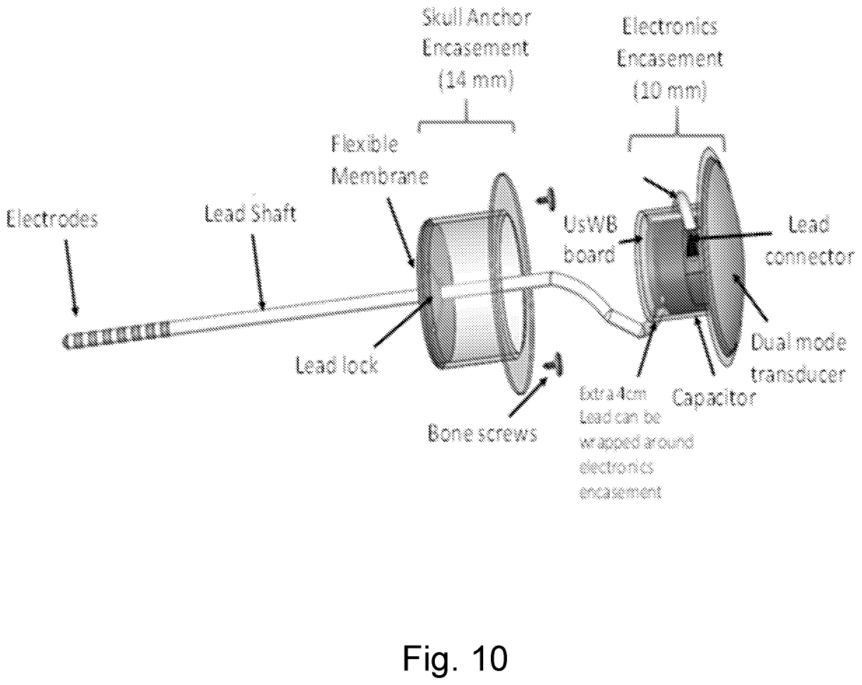

Aspects of engineering design solutions are described herein. The following design improvements may be implemented: (i) Dual frequency communication module with transmission and receiver amplification chain to cover a broader spectrum of frequencies (100 Hz-800 MHZ) may be integrated in UsWB board. Electronic/filtering elements may also be added. WSL may also have long range bi-directional communication of the WC (10 Hz-150 Khz frequency). The ceramic transducers may be coated in Parylene (30 μm) and biocompatible Polyurethane (80-100 μm, 35 Shore A durometer), any other polymer, metal, ceramic, epoxy, or composite material may be used to coat the transducer: (iii) An adjustable wave generator chip may be added to the WSL board to improve stimulation energy efficiency by powering down the MC when communication operations are not required based on the proposed duty cycles (0%, 0.4%) (iv) Mechanical design may be improved to include 8 electrodes, and software and hardware may be improved to allow for control of extra leads and directional stimulation: and (V) WSL may be encased in a two-parts to allow for easy replacement of electronics and variable length of lead shaft. As shown in FIG. 10 shows: a) a first encasement to anchor (with screws) WSL to skull, lock lead length after final adjustment and provide hemostasis through pierceable flexible membrane: and b) second encasement that releasably locks (through deformable clips) to the first encasement, that holds all electronics and is connected to the electrodes through the flexible shaft/lead. The shaft/lead may be attached to the electronics encasement through a connector that can be removed if electronics need to be replaced (lead/electrodes stay at stimulation site) or to connect to micro-electrode recording system during delivery. Difference in encasement diameters allows for a section of the flexible shaft/lead to be wrapped around the electronic encasement to provide variable length to reach the stimulation site (+2 cm). The electronics encasement may hold a single board (9 mm), capacitor (15 F), and dual frequency piezoelectric: (D) EG second transducer and corresponding electronics may be added to enable WSL to recharge and reprogram both WSLs in parallel. Casing design for electrical insulation and ergonomics may be improved with a cap/harness fixture developed to hold leads in place while recharging (10-20 minutes) for ease of use: (E) WC optimize drive/coupling circuitry for dual band transducer (150 Khz, 720 Khz) and add 512 Mbit RAM to the Xilinx Zynq-7000 System on Chip to enable future implementation of AI: and (F) smartphone application—add functionalities associated with dual-piezo EG to enable synchronous charging and reprogramming of both WSLs (parallel or series). Code may be robust, safe, and redundant to meet criteria for medical device software standards. Human factors associated with smartphone application interface may also be evaluated.

Translate FPGA/MC architecture into an application-specific integrated circuit (ASIC) custom chip, including all system functions. Complete engineering testing of all primary functions using ASIC based electronics with measurable durability of implant function at aggressive stimulation rates for at least 6 days in absence of recharge.

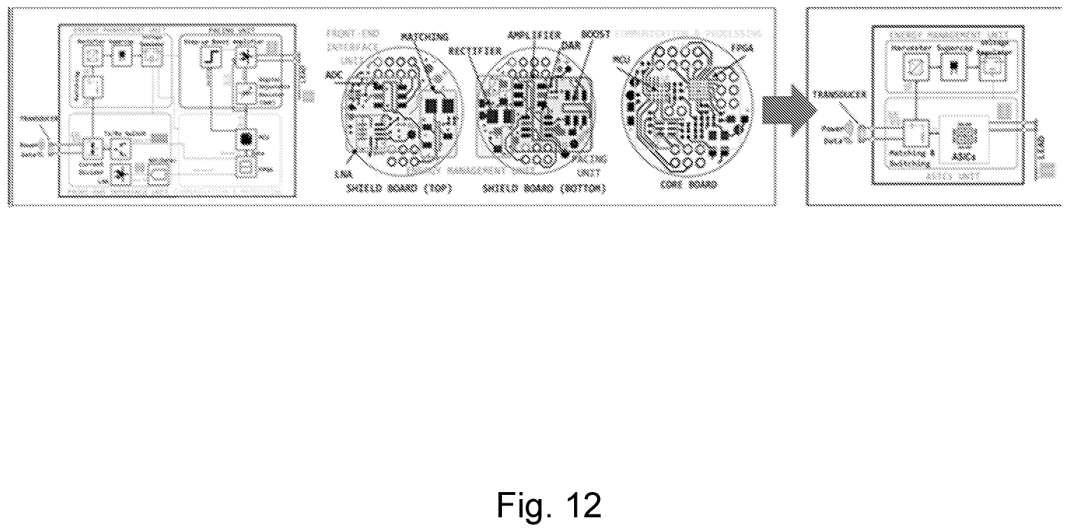

In various embodiments, FIG. 12 depicts various hardware architectures which may be used to implement systems described herein. For example, an ASIC chip may be designed and/or manufactured with the same functionality as FPGA/MCU based architecture depicted on the left-portion of FIG. 12, but with a smaller form factor. By using standard process, the WSL may be packaged in chip size 2 mm×3 mm to 4 mm×5 mm; and improving energy efficiency by 10×.

FIG. 12 depicts WSL micro-chip architectures, according to various embodiments of the present. In the left-portion of FIG. 12, an illustrative WSL is composed of FPGA & MCU architecture comprising: (i) a front-end interface connecting the transducer to the system: (ii) energy management unit tasked with harvesting and powering all units: (iii) a communication and processing unit incorporating MCU and FPGA to perform communication and computation tasks as well as generating pulses for the pacing/stimulation unit: and (iv) a pacing/stimulation unit adjusting the intensity of the generated pulses and driving them through a Digital Adjustable Resistor (DAR), a step-up boost and an amplifier. In the center-portion of FIG. 12, illustrative PCB layouts of dual board configuration are depicted, highlighting the different units and components on them. In the right portion of FIG. 12, a schematic showing simplicity of ASIC based configuration is depicted.

Aspects of ASIC design and manufacture are described herein. The manufacturing procedures to convert the proposed circuitry, although expensive and time consuming, are well known. Process flow is as follows: 1) Chip specification may be based on the electronic design used in SAI and energy efficiency performance requirements (RTL code is generated and test bench designed). 2) Modeling and mixed mode simulations (function & timing) may be used to confirm the functionality and logical behavior of the proposed circuit. 3) During RTL block synthesis the code may be translated into a gate-level netlist and synthesized into a database of the ASIC design. 4) The design may be partitioned into multiple functional blocks (hierarchical modules based on technical specifications and requirements) to determine what blocks can be based on previous design libraries. Several of the block unit libraries for our ASIC chip design may leverage known stimulation and modern telecom chips libraries, thus design efforts will focus on libraries associated to UsWB functionalities. 5) Functional block architecture and integration tested using multiple vectors from in silico tools. 6) Physical implementation of chip starts through block placement and ends with clock tree synthesis and routing: 6) During verification the Layout, the conformity to manufacturing rules, and the logical equivalence are checked: and 7) Final stage wafer processing, packaging, and testing is performed before delivering the design file to the semiconductor foundries for fabrication of the silicon chip. After fabrication of the new ASICs chip following the process above, they may be assembled onto small single board (≤9 mm diameter) for the WSL, EG and WC for minimum of 10× improvement in system energy efficiency.

Embodiments described herein provide devices, systems, communication protocols and wireless links, and methods for creating wireless networks of devices inside and outside the human body in order to treat patients without the complexity and complications associated with the use of implanted wired systems.

Many different networks of implantable devices to treat different etiologies may be designed using the UsWB technology to communicate and/or transmit energy. Recognizing that the versatility of the platform technology is significantly higher than a single medical application, a dual-level modular approach is the basis for the different networks. For the first level, implantable devices are designed and/or built following a modular approach (device level), by combining different functional units with the primary ultrasonic IoMT platform. Second, networked medical application are built by combining the functionalities of different implantable devices working in a coordinated fashion (network level). Such a modular approach reduces hardware changes associated with different applications by focusing on software/firmware changes on versatile hardware.

At the device level, the core building block unit, the “IoMT platform” is combined with functional units to define a functional network node. Typical functional nodes used as building blocks of medical devices (implants or wearables) are: (i) sensing node: (ii) actuation node: (iii) control node: and (iv) energy transfer/gateway node, or combinations thereof. In exemplary embodiments, a single medical device can be constructed with a single functional node, whereas in other exemplary embodiments a medical device may be defined by a plurality of functional nodes. Therefore, networked elements include both devices and/or functional nodes. A network under this definition can thus be described as a network of functional nodes or a network of medical devices, interchangeably, depending on the desired representation of the components.

At the network level, the re-programmability of each functional node (with perhaps limited hardware tuning), as well as their interconnection enabled by the common ultrasonic networking protocol stack, is used to create different networked applications based on interactions between similar functional nodes. Therefore, different networks of these functional nodes will allow for different therapies in different parts of the body as a limited breadth of actuation and monitoring functions with varying programming control can treat different pathological conditions.

A vast number of different networks of medical “things” may be created to treat or monitor patients with different pathologies. These may be simply a combination of a controller or gateway nodes with a sensor node for remote monitoring of patient health by measuring physiological parameters (e.g., a blood pressure sensor to monitor hypertension): or, can be as complex as networks with multiple and diverse actuators, sensors, control and gateway nodes that can be used to jointly monitor and treat a condition remotely. Most medical network applications can be grouped into two major categories: (i) intelligent monitoring and pacing networks: and (ii) monitoring and drug delivery control networks.

Several embodiments of implantable and non-implantable nodes have been designed for different applications such as: deep brain stimulation, cardiac pacing, cochlear/auditory device recharging and reprograming, intra-ocular pressure monitoring for glaucoma, wireless neonatal monitoring, brain neuro-stimulation, peripheral nerve stimulation, spinal cord stimulation, gastric pacing, artificial limb control, ventricular assist device control and recharging, monitoring or orthopedic implants, monitoring or artificial heart valves, glucose monitoring and insulin pump control, and controlled drug delivery among others.

In a preferred embodiment of the invention, network functional nodes are used for cardiac pacing and neurostimulation by themselves and/or in combination to treat different etiologies of human disease. In one exemplary embodiment a network of functional nodes is used to combine cardiac resynchronization therapy with neurostimulation in order to reduce mortality and improve quality of life in heart failure patients. In another exemplary embodiment a wireless bi-ventricular pacing network of functional nodes is used to reduce cardiac lead-based complications while monitoring cardiac pressure. In a further exemplary embodiment a network of functional nodes is used for wireless deep brain stimulation (DBS) in Parkinson's disease patients to reduce tremors and improve their quality of life. These networks all have in common functional pacing/stimulation nodes, sensor nodes, implantable control nodes, and external communication/recharging gateway nodes.

The invention provides a wireless medical device network comprising: a plurality of networked elements, wherein at least two of these elements communicate with each other by sending or receiving data using ultrasonic waves. In embodiments, the ultrasonic wave is a pulsed wave. In embodiments, the ultrasonic wave is a continuous wave.

The invention provides a wireless medical device network comprising: a plurality of networked elements, wherein data is encoded in the frequency domain of the ultrasonic wave. In embodiments, data is encoded in the phase domain of the ultrasonic wave. In embodiments, data is encoded in the amplitude domain of the ultrasonic wave.

The invention provides a wireless medical device network comprising: a plurality of networked elements, wherein data is encoded in the relative position of ultrasound pulses with respect to a reference. In embodiments, data is encoded using a time-hopping scheme in the ultrasonic pulses. In embodiments, a single bit of data is encoded in a single ultrasonic pulse. In embodiments, a single bit of data is encoded in multiple ultrasonic pulses.

The invention provides a wireless medical device network comprising: a plurality of networked elements, wherein the ultrasonic wave is generate by a unidirectional ultrasonic transducer. In embodiments, the ultrasonic wave is generate by a multidirectional ultrasonic transducer. In embodiments, at least one network element is implanted within the human body. In embodiments, at least one network element is wirelessly recharged using ultrasonic waves.

The invention provides a cardiac medical device wireless network comprising: a plurality of networked elements, wherein at least one element is an implanted device: and, wherein at least two of these elements communicate with each other by sending or receiving data using ultrasonic waves. In embodiments, at least one implanted device is used for pacing of a heart chamber: wherein at least one implanted control element is used to control a pacing element. In embodiments, at least one implanted control element is used to wirelessly recharge a pacing element using ultrasonic waves.

The invention provides a wireless medical device network comprising: a plurality of networked elements, wherein at least one of the implanted elements has the capacity to measure blood pressure. In embodiments, at least one of the elements can communicate wirelessly to the internet.

The invention provides a cardiac medical device wireless network comprising: a plurality of networked elements. In embodiments, the ultrasonic wave is a pulsed wave. In embodiments, a single bit of data is encoded in multiple ultrasonic pulses.

The invention provides a neurostimulation medical device wireless network comprising: a plurality of networked elements, wherein at least one element stimulates or modulates the nervous system. In embodiments, at least two of these elements communicate with each other by sending or receiving data using ultrasonic waves. In embodiments, at least one element is an implanted device. In embodiments, at least one device is implanted within the brain. In embodiments, at least one element is used for deep brain stimulation therapy. In embodiments, the ultrasonic wave is a pulsed wave. In embodiments, at least one device is connected to the peripheral nervous system.

The invention provides a neurostimulation medical device wireless network comprising: a plurality of networked elements, wherein the ultrasonic wave is a pulsed wave. In embodiments, at least one network element can increase or decrease cardiac function.

Radio-frequency (RF) electromagnetic waves, and specifically microwaves, are heavily absorbed by biological tissues fluid and other solids. As a consequence, (i) RF based transmission heats up tissues, which limits applications in delicate parts of the body such as the brain: (ii) signal absorption limits efficiency, thus requiring larger energy storage/batteries: (iii) tissues also significantly distort and delay RF signals, which causes data transmission to become less reliable: and (iv) absorption limits depth of signal penetration for data or energy transmission. In contrast, ultrasonic transmission of data and energy does not suffer from the draw backs, since mechanical waves are not absorbed to the same extent in biological tissues.

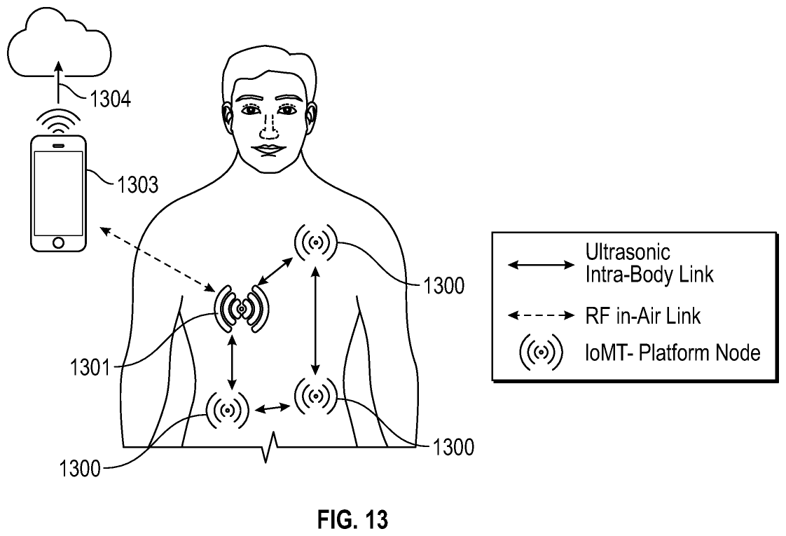

As shown in FIG. 13, in a preferred embodiment the nodes of the intra-body network 1300 communicate/link and/or send energy using ultrasonic waves while the nodes external to the body 1301 can use RF based communication links to communicate with other external elements. In exemplary embodiments, the extracorporeal nodes can still use ultrasonic waves to communicate through the air when such a link is desired. Using these external links the intra-corporeal network can communicate to the internet of things, the general internet, physical computers/servers/equipment, or the cloud. External communication links allow data to be stored or monitored remotely. In preferred embodiments, the intra-body network can also be controlled or re-programmed remotely using these data links. In exemplary embodiments, the data of one or more patients received from this networks can be processed using standard or artificial intelligence algorithms to improve patient treatment for the general patient population or provide patient centric tailored treatment.

Many different networks of implantable devices to treat different etiologies may be designed using the UsWB technology to communicate and/or transmit energy. There are several common types of implants that can be used in distinct networks to treat different forms of disease. Therefore, to reduce development timelines/cost and regulatory burden, proven modular hardware elements from one network are used in another to enable another applications. Therefore, a dual-level modular approach is the basis for the different networks. For the first level, implantable devices are designed and/or built following a modular approach (device level), by combining different functional units with the primary ultrasonic IoMT platform. Second, a networked medical application is built by combining the functionalities of different devices working in a coordinated fashion (network level).

At the device level, the core building block, “IoMT platform”, is used to create implants (and wearables) with specific functions by adding one or several functional units. Devices configured in this way are defined as a functional node. Typical functional nodes to used as building blocks of medical devices (implants or wearables are (i) sensing nodes: (ii) actuation nodes: (iii) control nodes: and/or (iv) energy transfer/gateway nodes.

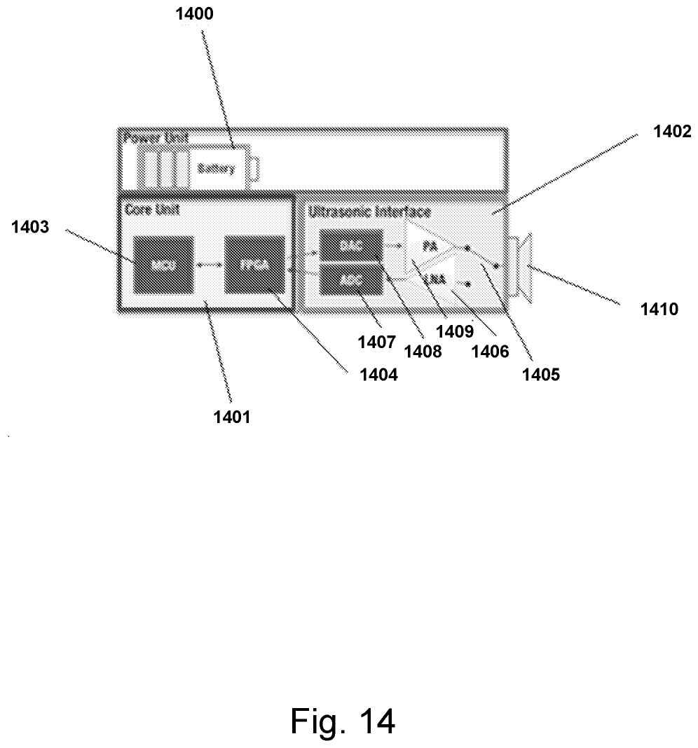

As shown in FIG. 14, in a preferred embodiment the basic IoMT platform is constructed from three primary units, although in some exemplary embodiments all these units may not be present. These primary units are: (i) power unit 1400, (ii) core unit 1401, and a (iii) ultrasonic interface 1402. In exemplary embodiments, the different components that make up these units above can be implemented in several circuit boards or electronic components. In some preferred embodiments, the IoMT is implemented in a multi-layer board that may contain all the units named above. In a further embodiment System-on-Chip (SoC) solutions will be used to integrate different hardware components of the IoMT platform inside a single chip.

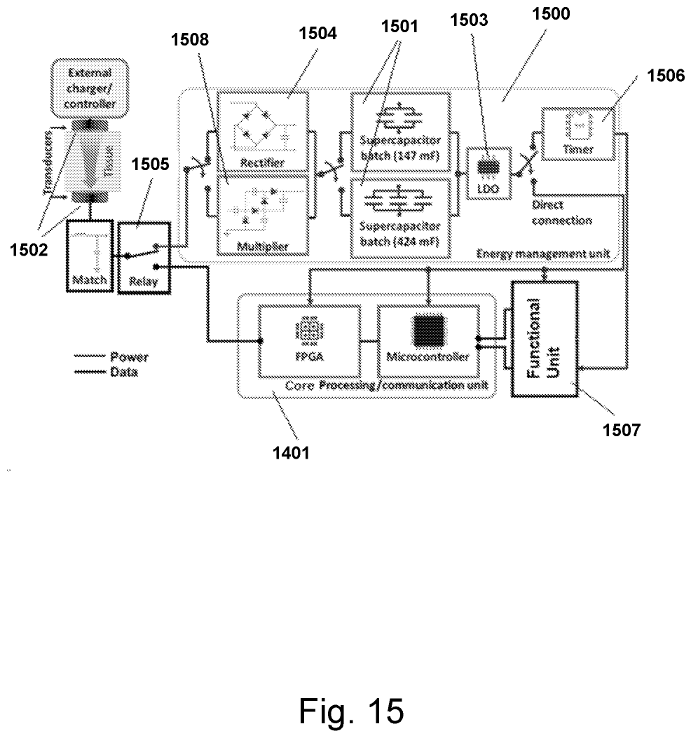

As shown in FIG. 15, in a preferred embodiment, the power unit will include both an energy harvester and/or energy storage/buffer when required, defining an energy management unit (EMU) 1500. Storage/buffer 1501 can take the form of a battery (standard or rechargeable), a capacitor, or an array of capacitors or any other voltage storing electronic element. The selection of the type of energy storage will depend on size requirements, life expectancy, and the risks associated with battery replacement.

In a preferred embodiment, the EMU 1500 includes an ultrasonic energy harvester that is used to receive ultrasonic waves from other node/nodes of the network and transform these waves into energy that can be used to power the receiving node. In this embodiment, different network nodes can received and/or send energy using ultrasonic waves. When sending energy, the EMU in combination with other units of the node can take alternating current (AC) signal, or a square wave, generated at ultrasonic frequencies (>20 kHz) to drive an electro-acoustic transducer 1502 that transmits power to another node. The transducer converts the electrical waveform to mechanical waves. When receiving ultrasonic energy, an acousto-electric transducer transforms the mechanical excitation back into an electrical AC signal. In an exemplary embodiment, the EMU can also contain a rectifier and/or a multiplier circuit, and a low dropout (LDO) regulator 1503 to limit the voltage delivered. The storage components (battery, capacitors, etc) 1501 need a direct current (DC) voltage to be recharged, which requires the presence of the rectifier 1504 whose role is to generate a DC voltage from an oscillating input. The core unit 1401 helps control part of the energy harvesting system as well as an ultrasonic transceiver for data communication. When utilizing the same transducer to send/receive data and energy, a switch 1505 is used to change from an energy sending/receiving phase to a communication phase in the operation cycle of the transducer. During the first phase, the platform is remotely recharged via ultrasonic transcutaneous energy transfer (UTET). During the second phase, the harvested energy is used to power the circuitry to activate processing, sensing, actuation, and communication functions. In specific embodiments, the platform actually sends energy during the first phase and second phases, such as in the case of a IoMT platform as part of a control node. The phase switch can work using prescribed times from a timer, or can be controlled by the core unit elements 1401. In exemplary embodiments of the energy harvesting system, different transducers are used to send/receive data and energy, thus not requiring the switch controlled 1505 dual phase cycle described above. Additional elements as a secondary timer 1506 to control switching between data processing in the core unit 1401 and powering the functional unit 1507 of the functional node, can be included, to improve energy efficiency. A multiplier circuit 1508 can also be included to help with signal processing and/or amplification.

The core unit 1401 may include a stand-alone microcontroller, or a combination of a microcontroller (MCU) 1403 and reconfigurable hardware, such as a field-programmable gate array (FPGA) 1404; or even a stand-alone FPGA. In exemplary embodiments, other type of reconfigurable hardware can be used like application-specific integrated circuits (ASICs). Depending on the processing requirements and desired size, any of these options may be used. In a preferred embodiment where the MCU and FPGA are combined, their combination results in hardware and software reconfigurability with very small packaging and low energy consumption. The miniaturized FPGA 1404 hosts the physical PHY layer communication functionalities. The MCU 1403 is in charge of data processing and of executing software-defined functionalities to implement flexible and reconfigurable upper-layer protocols. These upper-layer protocols may include in some embodiments non-time critical MAC functionalities, network, transport and application, among others. In the exemplary embodiment, the software is split between FPGA 1404 and MCU 1403. The FPGA 1404 implements the PHY layer communication functionalities, as well as interfaces to connect the FPGA 1404 chip with the MCU 1403 and the peripherals. In the exemplary embodiment the MCU software design is based on the uTasker real-time operating system (RTOS) that supports timers and interrupts for sensing and transmitting data and executes the upper layer networking protocols. The IoMT platform software framework also provides a set of primitive functions to be used as building blocks to develop specific data processing applications. To someone skilled in the art is it understandable that all software could be implement in any of the components that may be included in the core unit dependent on the algorithm design and processing requirements. In the preferred embodiments the functionalities are implemented to minimize the system energy consumption by leveraging uTasker primitives to access different power states. Specifically, an energy management module is able to (i) adjust at runtime the core clock frequency and low-power mode according to application requirements, and (ii) implement automatic wake-up functionalities. This implementation will allow the MCU current consumption to be reduced from its values in a RUN state down to lesser values in very-low-leakage-state, with intermediate states that trade current consumption for wake-up time.

The ultrasonic interface 1402 in preferred embodiments will be common to different types of nodes in the same network, thus enabling internetworking: depending on the power unit type, it may also have an interface for energy harvesting. The ultrasonic interface enables wireless connectivity and consists of a receiver (Rx) and a transmitter (Tx) chain. Depending on the number of ultrasonic transducers, the Rx and Tx can work in parallel or may need to be switched 1405 to work in series in controlled cycles over time. The Rx chain includes a low-noise amplifier (LNA) 1406 and an analog-to-digital converter (ADC) 1407 to amplify and digital-convert received signals, while the Tx chain embeds a digital-to-analog converter (DAC) and a power amplifier (PA) to analog-convert and amplify the digital waveform before transmission.

The ultrasonic interface can use a single or a plurality of transducers. When a single ultrasonic transducer is used to operate several send/receive cycles, these cycles will be gated in time. In some embodiments both energy and data could be send in the same acoustic signal, although with limitation in the data transmission rate. In exemplary embodiments, multiple transducers can to send and or receive different signals. In exemplary embodiments, different transducers can be used to separate the energy and data transmission functions or different transducers can be used to send signals in different directions. In exemplary embodiments, unidirectional transducers may suffice, but when directional tolerances are important so that the beam reaches efficiently another node, a multidirectional or omni-directional transducer should be used. Directionality of the transducers is achieved by an array design (array of unidirectional piezos pointed in different directions) or by the shape of the ultrasound emitting element or piezo (cylindrical, semi-spherical, spherical, etc). Both, piezoelectric ultrasonic transducers (crystal, ceramic, polymers, organics and composites) or capacitive ultrasonic transducers can be used in the ultrasonic interphase unit. In exemplary embodiments, capacitive transducers include those micro-machined using MEMs technologies and well as others which use more traditional manufacturing methods.

There are several exemplary embodiments that describe the ultrasonic waveforms used to send or received energy and/or data using UsWB. In some embodiments continuous ultrasound waves can be used while in others pulsed ultrasound waves may be preferred. In preferred embodiments using pulsed ultrasonic waves, discrete digitally modulated pulses of ultrasound are emitted from the transducer (discrete ultrasound wave pulse packages). In embodiments where data is sent through continuous waves, data can be encoded in the frequency or phase (frequency shift keying or phase shift keying) or amplitude domain (amplitude shift keying) or in a combination (quadrature amplitude modulation). In other embodiments data can be encoded in the relative position of ultrasound pulses with respect to a reference (pulse position modulation). When using pulsed ultrasound waves, data can be encoded in the frequency or amplitude domain, or in the relative positions of pulses in the ultrasonic wave. In a preferred embodiments, a time-hopping scheme is used to encode the data in the ultrasonic pulses. In some exemplary embodiments each bit of data can be encoded in a single pulse, while in preferred embodiments a single bit can be encoded within the signal structure of multiple pulses. In certain embodiments each bit can be represented with multiple pulses whose polarity or position can be modulated following a binary spreading code. The spreading code can be obtained through a pseudo-random generator, or it can follow a known and pre-defined pattern.

The core building block, the IoMT platform, can be combined with a functional unit 1507 to create a functional node as described above. Possible functional units may have the largest range of variability and will determine the nature of the node. For example, for control nodes, the specification in its processing (core unit) and communication elements (ultrasonic interface) can define its function completely. In contrast, for energy transfer/gateway node the specific data interface with the external environment (e.g., WiFi, Bluetooth) and the type of energy transfer interface will define its range and specific function. In some embodiments, sensing nodes or actuation nodes (e.g., pacers) may tend to have a simple microcontroller as core unit: whereas control nodes and energy transfer/gateway nodes may require a more reconfigurable hardware so the same unit can be used in several different types of networks with very limited changes to hardware. Therefore, a large variability of component architecture can be used in different functional nodes depending on specification.

For sensing nodes, the specific physical sensor (electrical, acoustic, EKG, pressure, temperature, voltage/current, flow, chemical, Gas, Ph+ sensor, photosensor, accelerometer, etc) will define the functionality. Further, a specific sensing node can find many different medical applications in different parts of the body. For example, a pressure sensor can be used in cardiovascular (heart failure, Hypertension), ophthalmological (glaucoma), and spine (disk compression) applications, among others.

In some applications sensors nodes may be used in wearables or outside the body to create wireless network links through the air in environments where sensing of the human body is required and RF based links are not possible or not preferable. In a particular embodiment of UsWB transmission through the air, in the nursery or neonatal intensive care unit wired connections to sensors on the child's body or surrounding environment may be exchanges for wireless UsWB links to diminish concerns of having a high density RF environment surrounding a neonate or young child. This link can interface with an external control gateway to send data to a computer or the internet.

Actuation functional units also present a large range of variation, but in general for the human body the primary expected actuators are (i) mechanical/artificial limbs/prosthesis, (ii) electro stimulation/pacing. (iii) cameras, (iv) acoustic, and (v) drug delivery/pumps. Many applications may work in absence of a sensor node like when using a control or gateway node to recharge and/or reprogram and/or adjust a cochlear implant or auditory support device. In such an embodiment, the controller or gateway node may be located or embedded in an acoustic friendly environment like a gel pillow to recharge the auditory device when the patient is at rest, or as required.

Although there is a large range of possibilities and scopes, it should also be understood that the nervous system and the heart work within a very similar electro-potential range. Therefore, reprogrammable-pacing/stimulation actuation functional units for pacing the myocardium, the brain or nerves, can have in many cases identical/similar electronics, with differences mostly associated to casing, software, and anchoring elements. Therefore, exemplary embodiments of pacing/stimulation actuation functional unit may generate many types of functional nodes for several parts of the body for heart, brain, and nerve stimulation. Exemplary embodiments of this functional nodes also includes pacing of the gastric system (stomach, intestine) that is accomplished using a similar electro-potential pacing actuation node. Increased energy capacity for pacing may also allow for skeletomuscular stimulation or prosthesis control.

The preferred embodiment of treatment specific modular networks is associated to cardiovascular disease and neurostimulation/neuromodulation. In these networks implantable pacing or stimulation nodes may be controlled and powered/recharged by an implantable control node, that itself can be controlled, recharged and linked to an external network using an energy transfer/gateway node. In some simpler embodiments the pacing or stimulation nodes can be recharged and controlled directly by the energy transfer/gateway node in absence of the implanted control node.

In a primary embodiment of UsWB enabled pacing networks, ultrasonic energy transfer and UsWB communication technologies can be used to reduce complications associated with wired pacing leads (infection, lead failure, pain, etc), reduce battery exchange rates, and hospital visits through remote monitoring in patients that need single chamber or multi-chamber cardiac pacing. A cardiac pacing network to pace a single chamber of the heart or multiple chambers can be constructed from pacing functional nodes, a control node and an energy transfer/gateway node. This network may also include cardiac pressure sensing within the pacing node or as an independent pressure-sensing node. The exemplary embodiments for this network include single ventricle pacing or bi-ventricular pacing, although other applications may include or be limited to atrial pacing of a single or both atria.

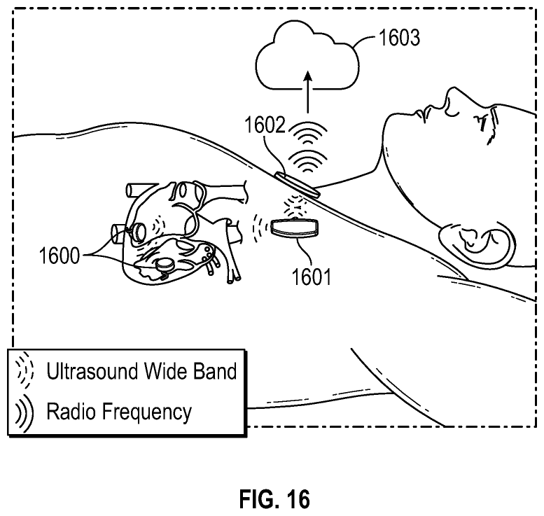

In a preferred embodiment for a bi-ventricular pacing UsWB network, three different types of functional nodes are defied as shown in FIG. 16: (1) reprogrammable wireless pacing (RWP) nodes with vascular pressure monitoring 1600, (2) rechargeable system (RS) control 1601 node, and (3) external recharging and communications (ERC) node 1602. All the elements in this network communicate with each other through proprietary Ultrasonic Wide Band (UsWB) technology. The RWP nodes and the RS control node are wirelessly recharged using ultrasonic energy transmission. UsWB platform is capable of sending energy and data via ultrasonic waves through tissue, bone, and fluids at penetration depths significantly higher than RF waves (up to 30 cm depth) and with better reliability (bit error rates better than 106). Since increasing energy efficiency results in reduced energy storage requirements, UsWB also enables miniaturization of medical devices. The RWP nodes 1600 are implanted using minimally invasive (catheter based endovascular methods) in both ventricles, the RS controller 1601 is implanted in a subcutaneous pocket in the chest of the patient, and the ERC node 1602 is a wearable which is in contract with the skin. The ERC node 1602 is removable and in preferred embodiments is attached to the skin using a glued patch. The contract area between the ERC node casing and the skin has ultrasonic coupling material.

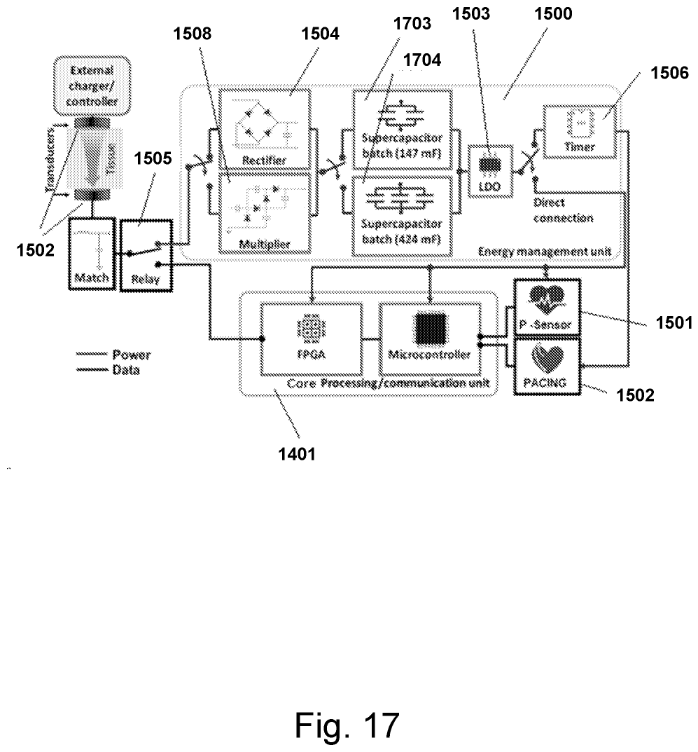

As shown in FIG. 17 The components of the RWP nodes are similar to those of the generic functional node described in FIG. 15 with the addition of two functional units, a pressure sensor 1701 and an electric sensor and pacing unit 1702. The RWP nodes are recharged using ultrasonic waves and therefore include the EMU 1500 with harvesting capabilities. RWP nodes 1600 have reprogrammable electronic hardware that can be easily adapted to pace cardiac muscle and/or other structures. RPWs are anchored to the myocardium with a conductive metallic coil (titanium, stainless steel, other conductive alloys) or any other conductive anchor (hooks, barbs, arms), and charged and controlled using ultrasonic communication and energy. The RPWs are able to communicate with each other to provide Cardiac Resynchronization Therapy (CRT) while in parallel storing energy for continued pacing for several hours or days using a capacitor bank. In this embodiment even in absence of the ultrasonic link to the control node a patient is given enough time to reach a hospital (safety feature) compared to current wired technology that immediately loses pacing capabilities after lead failure. In a preferred embodiment super-capacitor energy storing bank (2 or more capacitors) and rectifying circuit are used to have redundancy in a zero failure environment. One capacitor 1703 will be dedicated to pacing and the other 1704 will always be fully charged (safety). Although in some exemplary embodiments pressure sensors (MEMs or non-MEMs) may require separate implants, in an exemplary embodiment a MEMS pressure sensor 1701 is integrated into the RPWs to actively monitor left and right ventricular pressures. The core unit can include a FPGA 1404, a MCU 1403 or an ASICs or any combination thereof. In a preferred embodiment a FPGA is used to drive the logic of the MEMs pressure sensor and control all functions within RPW lead in combination with the MCU 1403. The electric sensor 1702 is used to track real time the electrical behavior of the implant site in order to respond to changes in the rhythm of the heart. These responses from the RWP to changes in the rhythm of the heart can occur in a closed loop with its own core unit, or in a closed loop combination with data provide by the pressure sensor on any of the other of the implants. Pacing responses can also be re-programmed by a clinician on site or remotely using the ERC patch 1602.

In a preferred embodiment, the pacing electronics will be encased within a Polyether ether ketone (PEEK) casing and wrapped with medical-grade porous Dacron cloth to promote rapid tissue in-growth reducing thrombogenic risk. In exemplary embodiments the casing can be constructed of other polymers or metals, and may include a non-thrombogenic surface. In an exemplary embodiments the casing will have a single silicone (or other polymer) window above and in contact with the MEMS pressure sensor in order to transmit the external pressure to the sensor without or with reduced interference from the case itself.

Rechargeable system (RS) control node 401 consist of a reprogrammable controller that can coordinate and re-program other implantable elements of the network through the ultrasonic interface, the basic architecture is that shown in FIG. 3 but doesn't include a functional unit 307 since this node doesn't require any sensory or actuator functionalities. The controller is also able to transmit energy from its position just below the skin to other elements implanted further away from the surface. The battery of the controller is recharged through the skin using ultrasonic power transmission, therefore requiring and EMU 300 with harvesting capabilities. The controller will also be reprogrammed/controlled from outside the body and serve as an interface for off-site remote monitoring. The basic architecture of the controller includes multidirectional ultrasonic transducers and a high-capacity rechargeable battery. In an exemplary embodiment a single omnidirectional ultrasonic transducer may be used. In a preferred embodiment two transducers may be used, one directed at the RWP nodes 400 and another toward the skin to communicate with the ERC patch 402.

External recharging and communication (ERC) 402 node/patch will recharge the RS control node through ultrasonic transcutaneous energy transfer when needed, and act as gateway to interconnect the intra-body network to the Internet. This energy transmission/gateway node is external element (wearable) to be used when needed to recharge or reprogram the network. The ERC node/patch has the same primary components as the IoMT platform shown in FIG. 14 with the addition of a functional unit to interphase with the internet or internet of things through WiFi and or Bluetooth BLE. In an Exemplary embodiment the ERC node can be recharged wirelessly, although in a preferred embodiment the ERC node will have a connector port to hookup a recharging cable that can be plugged to the wall. In some exemplary embodiments the ERC node may also have a USB port or Ethernet cable connection slot so that it can be updated or directly connected to a wired modem.

In exemplary embodiments the bi-ventricular pacing network shown in FIG. 16 will be capable of demand pacing and/or rate-responsive pacing. When in demand mode the RWP nodes will monitor the patient's heart rhythm and send electrical pulses if heart rates drops below a predetermined threshold. When in rate-responsive mode the RWP nodes will speed up or slow down your heart rate by sending pacing pulses depending on how active the patient is. In exemplary embodiments of rate-responsive pacing, the elements of the network will monitor other variables of your activity level through sensors in the RWP nodes and RS controller. These additional sensor units will monitor breathing through motion detectors or accelerometer, blood temperature through sensors in the RWP nodes, and other factors independently or as a group, that drive pacing algorithms. Such algorithms will work in a closed loop within the intra-body network and should not require physician input during real time implementation.

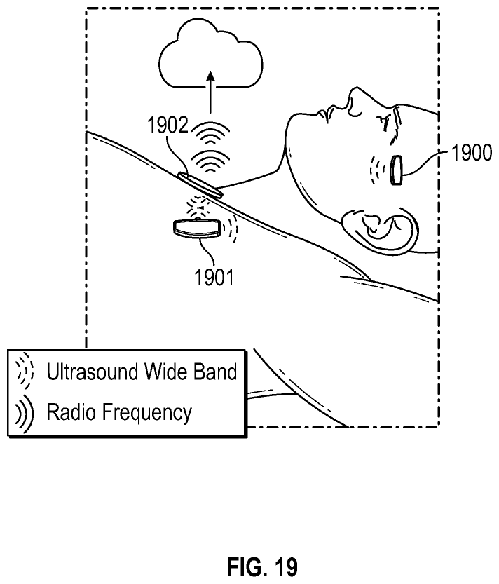

In another preferred embodiment of UsWB enabled pacing/stimulation networks, ultrasonic energy transfer and communication technologies can be used to reduce complications associated with wired pacing leads (infection, lead failure, pain, etc), reduce battery exchange rates, and hospital visits through remote monitoring in patients who need Neuromodulation/neurostimulation therapies. As shown in FIG. 6, many different pathologies may be treated using neurostimulation or neuromodulation both in the central and peripheral nervous systems. In a preferred embodiment a network of wireless leads nodes, controller node, and external network energy transfer/gateway node, can be used to treat tremors in the Parkinson's disease population through deep brain stimulation (DBS). Similar to the bi-ventricular pacing network the DBS wireless network shown in FIG. 19. consists of three main types of components/nodes (1) reprogrammable stimulation nodes (RSN) 1900, (2) rechargeable system (RS) control node 1901, and (3) external recharging and communications (ERC) node 1902. The ERC node/patch and the RS control node have the same architecture as that described above for the bi-ventricular pacing network, with basic differences in the energy storage/transfer capacity for these two nodes. For the DBS network, the RS control node is implanted in subcutaneous pockets in either the chest of the patient, the back near the neck between the shoulder-blades or subcutaneously in the back of the head. ERC node for this DBS network is a wearable that is in contract with the skin as required. During its function it will be located externally on the skin just above the RS control pocket. The ERC node is removable and in preferred embodiments will be attached to the skin using a glued patch. The contract area between the ERC node casing and the skin has ultrasonic coupling material.

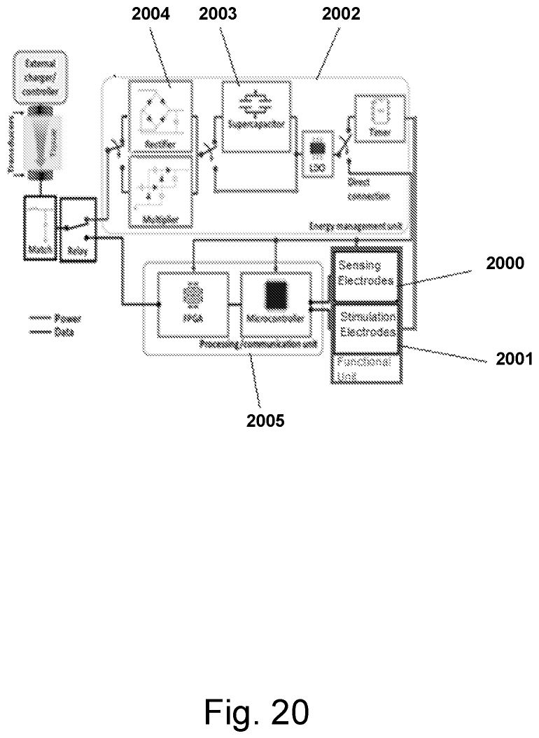

As shown in FIG. 20, the components of the RSN nodes are similar to those of the generic functional node described in FIG. 15 with the addition of two functional units, an electrical sensor unit 2000 and stimulation unit 2001 for multi-contact stimulation. The RSN nodes have reprogrammable electronic hardware that can be easily adapted to unilaterally or bilaterally stimulate the subthalamic nucleus (STN) and/or other structures. The nodes are anchored in the brain and use one or more conductive (metallic) electrode ring contact points. In a preferred embodiment 4 to 8 contact electrode rings are used per RSN node. In a preferred embodiment these ring are made of a platinum Iridium alloy. The RNS node is recharged using ultrasonic waves and therefore include the EMU 2002 with harvesting capabilities. RNS nodes 1900 have reprogrammable electronic hardware that can be easily adapted to deep brain stimulation or other types of neurostimulation. The RNS nodes are able to communicate with each other to provide bilateral DBS while in parallel storing energy for continued stimulation for several hours using a capacitor 2003. In some exemplary embodiments the RNS node will not need long term energy storage (capacitor or battery) as it may use directly the energy being transmitted to it with just transient energy buffering. In a preferred embodiment, energy long-term storage or transient energy buffering need a rectifying circuit 2004 to convert AC voltage from the transducer to circuit usable DC voltage. The core unit 2005 will include a FPGA, a MCU or an ASICs or any combination thereof. In a preferred embodiment a FPGA is used to control all functions within RNS node in combination with the MCU. The electric sensor 2000 is used to track real time the electrical behavior of the implant site in order to respond to abnormal firing of neurons directly associated to tremors. The stimulation ring array 2001 will emit electrical signals (square wave, sinusoidal wave, or other wave geometries) with frequencies between 3-250 Hz (mode dependent) to mitigate tremors in the patient. The processing, communication, and rapid response capabilities of the RNS nodes also allow the system to sense from the electrode rings and then alter the stimulation output based upon that input—a so-called “closed-loop” system, which is the basis for a new technology paradigm in DBS. Although unidirectional ultrasonic transducers may be used: in preferred embodiments Semi-spherical piezo electric transducers and/or omni-directional piezoelectric transducers are used by the RNS nodes to reduce directional sensitivity when emitting or receiving the ultrasonic waves. The casing is constructed of electric insulating materials, in a preferred embodiment the casing will be constructed of polyurethane. Use of a single RNS node in the network allow for uni-lateral stimulation while two RNS nodes are used for bilateral stimulation. DBS sites in which the RNS transducers can be used include the subthalamic nucleus, globus pallidus internus, pedunculopontine nucleus, and ventral intermediate nucleus of the thalamus among others.

In an exemplary embodiment of wireless UsWB treatment networks, elements from the bi-ventricular cardiac pacing network and the DBS network can be combined to help patients with heart failure (HF). Recent declines in mortality of HF patients have been attributed to the use of cardiac resynchronization therapy (CRT). Studies have also shown that remote monitoring of cardiac implantable electronic devices improves HF patient survival and is also associated with reductions in hospitalization and health care costs. CRT combined with inotropes has been shown to improve heart function leading to reduced mortality. Neurostimulation of the sympathetic and parasympathetic nerves can control stroke volume (sympathetic to increase, parasympathetic to decrease contraction), thus provide similar mechanistic results as the use of inotropes. Therefore, clinical evidence suggests that an intelligent CRT device that can be monitored and reprogramed remotely, and that can be combined with neuromodulation to improve heart function would have a profound impact on treatment of HF patients.

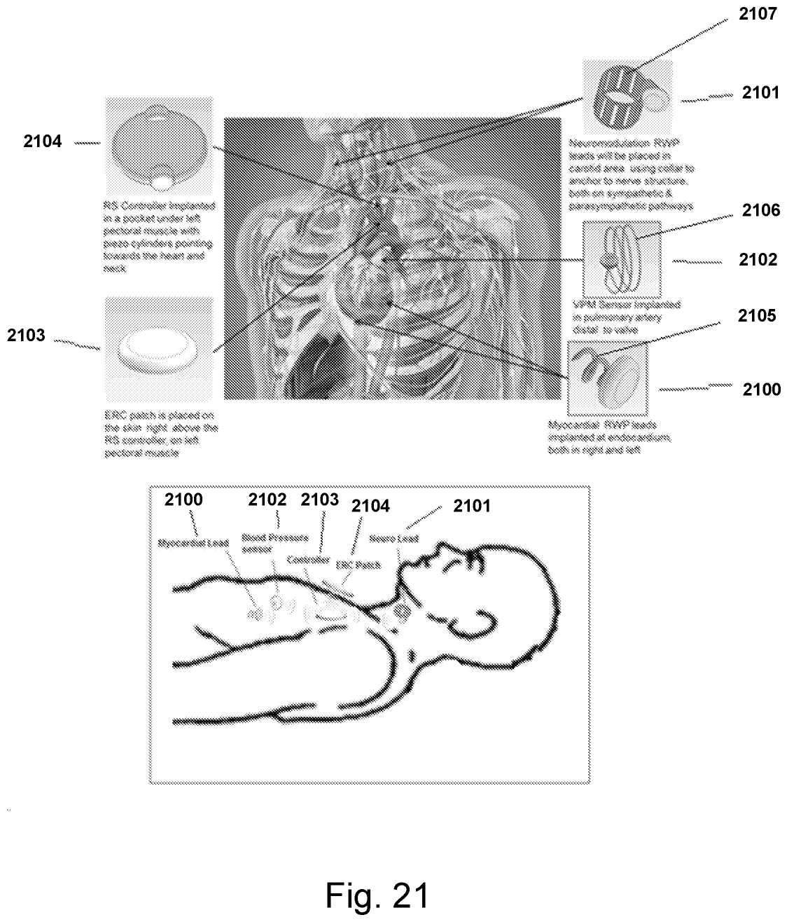

A modular neuromodulation/CRT network with pressure monitoring capabilities will not only improve patient outcomes but also reduce hospitalization rates and healthcare economics. The primary elements of the network for treatment of HF in patients through pacing/neuromodulation, as illustrated in FIG. 21, are: (i) Reprogrammable cardiac wireless pacing (RCWP) node 2100: (ii) reprogrammable neuro wireless pacing (RNWP) node 2101: (iii) vascular pressure monitoring (VPM) node: (iv) rechargeable system (RS) control node: and (v) external recharging and communication (ERC) node. All the elements in this network communicate through the Ultrasonic WideBand (UsWB) technology: and are recharged using ultrasonic energy transmission (excluding recharging of the ERC node).

The ERC node and the RS control node have the same architecture as that described above for the bi-ventricular pacing network and DBS network, with basic differences in the energy storage and transfer capacity of these two nodes. For the neuromodulation/CRT network, the RS controller is implanted in subcutaneous pockets in either the chest of the patient. In an exemplary embodiment, a second RS controller may be required in the network if the distance between the RCWP and RNWP is too long and requires a network bridge. Since the RS control node in this network is required to communicate with the RCWP nodes, the RNWP nodes, and the ERC patch, several ultrasonic transducers are required. These transducers may be unidirectional, multidirectional or omnidirectional as required by implant location geometry. ERC node/patch for this DBS network is a wearable which is in contract with the skin that during its function will be located externally on the skin just above the RS control pocket. The ERC patch is removable and in preferred embodiments is attached to the skin using a glued patch. The contract area between the ERC node casing and the skin has ultrasonic coupling material.

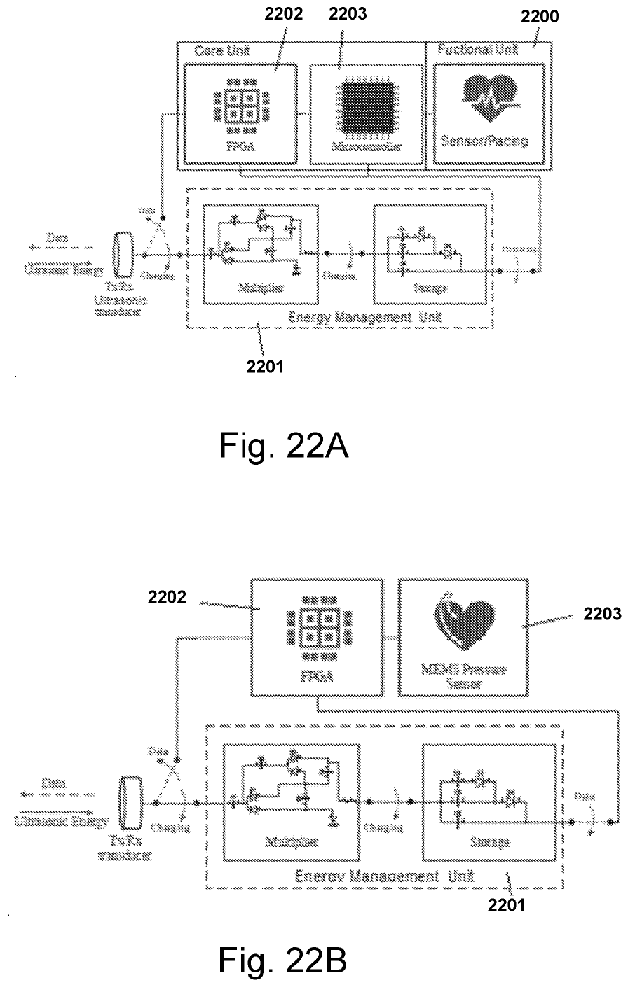

As shown in FIG. 22A. Reprogrammable wireless pacing (RCWP) nodes architecture is similar to those of the generic functional node described in FIG. 15 with and additional electric sensing and pacing functional units 2200. The RCWP nodes are recharged using ultrasonic waves and therefore include the EMU 2201 with harvesting capabilities. Reprogrammable wireless pacing RCWP nodes 2100 have reprogrammable electronic hardware and are anchored to the myocardium with a conductive metallic coil 2105 (titanium, Stainless Steel, Other conductive alloys) or any other conductive anchor (hooks, barbs, arms), and are controlled using UsWB. The RCPWs are able to communicate with each other to provide CRT while in parallel storing energy for continued pacing for several hours or days using a single capacitor or capacitor bank. In a preferred embodiment, even in absence of the ultrasonic link to the RS control node, a patient is given enough time to reach a hospital (safety feature). In a preferred embodiment, a capacitor energy storing bank (2 or more capacitors) and rectifying circuit are used to have redundancy in a zero failure environment. The core unit can include a FPGA 2202, a MCU 2203 or an ASICs or any combination thereof. In a preferred embodiment a FPGA is used to control all functions within RCPW node in combination with the MCU. The electric sensor 2200 is used to track real time the electrical behavior (EKG) of the implant site in order to respond to changes in the rhythm of the heart. These responses from the RWP to changes in the rhythm of the heart can occur in a closed loop with its own core unit, or in a closed loop combination with data provide by the VPM node or any of the other network elements. Pacing responses can also be re-programmed by a clinician on site or remotely using the ERC node 2103. In a preferred embodiment the pacing electronics will be encased within a Polyether ether ketone (PEEK) casing and wrapped with medical-grade porous Dacron cloth to promote rapid tissue in-growth reducing thrombogenic risk. In exemplary embodiments the casing can be constructed of other polymers or metals, and may include a non-thrombogenic surface.

As shown in FIG. 22B, the vascular pressure monitor (VPM) node is a discrete implant in the neuromodulation/CRT network. The core unit for the VPM node may include a FPGA 2202, and/or MCU and/or ASICs. In a preferred embodiment the core unit only includes a FPGA 2202 in order to reduce implant size. The VPM node is recharged using ultrasonic waves and therefore include the EMU 2201. The ultrasonic interface of the IoMT platform, together with the energy management unit (Energy harvesting unit and capacitor-based energy storage) allows for wireless node function. In a preferred embodiment only transient energy storage (small capacitor or energy buffer) is required. Ultrasonic powering and communication capabilities are connected to a pressure sensor, in a preferred embodiment a MEMs pressure sensor is used. The VPN node uses a polymeric or resin casing to isolate the electronics from contacting blood. In a preferred embodiment, non-thrombogenic surfaces are used on the external faces of the casing. In an exemplary embodiment a window of elastomeric, linear elastic and or thinned out polymer is used to transfer the pressure load from the fluid to the MEMs sensor. Many exemplary embodiments can use, hooks, rods, suture, coils, arms, cylindrical stents among other as anchoring elements. In a preferred embodiment, a self-expanding Nitinol anchor wire is used to attach the VPN node to the pulmonary artery 2106. The VPN node will be deliver to its implant site using catheter based endovascular methods.

The components of the RNWP nodes are similar to those of the generic functional node described in FIG. 15 with the addition of two functional units, an electrical sensor unit and stimulation unit for single contact and/or multi-contact stimulation. The RNWP nodes have reprogrammable electronic hardware that can be easily adapted for unilateral or bilateral (multipoint) stimulation of sympathetic and parasympathetic nerves at the carotid level. The nodes are anchored to the carotid nerves and use one or more conductive (metallic) electrode ring contact points located on the inner surface of the anchoring collar 2107. In a preferred embodiment these rings are made of a platinum Iridium alloy. The RNWP nodes are recharged using ultrasonic waves and therefore include the energy management unit with harvesting capabilities. The RNWP nodes are able to communicate with each other to provide bilateral carotid nerve stimulation while in parallel storing energy for continued stimulation for several hours/days using a capacitor or capacitor bank. In a preferred embodiment energy storage elements need a rectifying circuit 2004 to convert AC voltage from the transducer to circuit usable DC voltage. The core unit will include a FPGA a MCU or an ASICs or any combination thereof. In a preferred embodiment a FPGA is used to control all functions within RNWP nodes in combination with the MCU. The electric sensor is used to track real time the electrical behavior of the implant site in order to stimulate at different frequencies and times the parasympathetic and sympathetic nerves to increase or decrease heart contractility/stroke volume. The stimulation ring array will emit electrical signals (square wave, sinusoidal wave, or other wave geometries) to change contractility of the heart/stroke volume. The processing, communication, and rapid response capabilities of the RNWP nodes also allow the system to sense from the electrode rings and then alter the stimulation output based upon that input—a so-called “closed-loop” system. Although unidirectional transducers may be used, in preferred embodiments multidirectional transducers, semi-spherical piezo electric transducers and/or omni-directional piezoelectric transducers are used by the RNWP nodes to reduce directional sensitivity. The casing is constructed of electric insulating materials, in a preferred embodiment the casing will be constructed of a polymer or resin.

The RS control node will coordinate CRT with neuromodulation to ensure synchronized changes in heart rate and contraction. Interaction with the VPM node will allow to improve therapy according to changes in hemodynamics. Use of the ERC node to send pacing, neuromodulation, and vascular pressure data remotely will allow clinicians to closely monitor patients reducing the need on in-hospital visits.

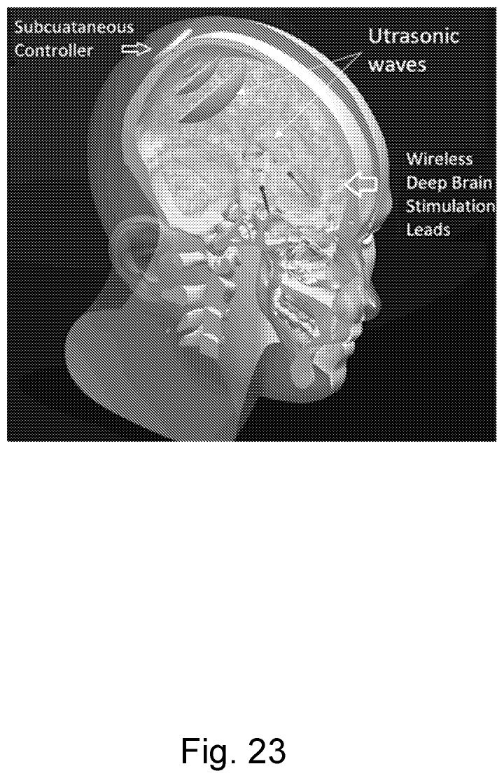

FIG. 23 is a diagram of an embodiment of a deep brain stimulation system wherein RWPs are fully embedded in the brain and controlled by an implantable extracranial controller.

It is to be understood that the above-described devices and methods for treatment of patients using ultrasonic wireless networks of implantable and non-implantable devices may include additional or alternative steps and aspects, based on the foregoing description relating to the various nodes/devices and tools described herein. Accordingly, as a result of the structure and functionality of the above-described networks, devices/nodes and tools, one skilled in the art would appreciate the different methods in which they may be utilized.

Many modifications and other embodiments of the present invention will come to mind to one skilled in the art to which the invention pertains upon having the benefit of the teachings presented herein through the foregoing descriptions and the associated drawings. Therefore, it is to be understood that the present invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A wireless medical device network comprising:
one or more implantable devices for intracranial use within a subject, each of the one or more implantable devices comprising at least one stimulation means, one or more circuits for collecting system data, a receiver, and a transmitter for communications using ultrasonic waves;
a wearable controller external to the subject, the wearable controller configured to:
communicate with the one or more implantable devices using the ultrasonic waves and obtain the system data;
analyze the system data to determine whether the subject is experiencing or is expected to experience an adverse medical condition; and
communicate with the one or more implantable devices using the ultrasonic waves to apply a stimulation to treat the adverse medical condition.

2. The wireless medical device network of claim 1, wherein the adverse medical condition is Parkinson's Disease.

3. The wireless medical device network of claim 2, wherein:
the wearable controller comprises an accelerometer/gyroscope (AG) that collects tremor data and is further configured to:
determine whether the tremor data is indicative of Parkinson's tremors or normal body function; and
responsive to a determination that the tremor data is indicative of Parkinson's tremors, cause the one or more implantable devices to apply a stimulation to the subject.

4. The wireless medical device network of claim 1, wherein wearable controller is configured to use a convolutional neural network (CNN) to analyze the system data.

5. The wireless medical device network of claim 1, wherein the one or more circuits comprises a field-programmable gate array (FPGA) and microcontroller unit (MCU).

6. The wireless medical device network of claim 1, wherein the one or more circuits comprises an application-specific integrated circuit (ASIC).

7. The wireless medical device network of claim 1, wherein:
the one or more circuits are configured to:
continuously collect system data;
determine, using a first convolutional neural network (CNN) that the system data comprises an abnormal pattern; and
in response to the abnormal pattern being detected, initiating transmission of the system data to the wearable controller; and
the wearable controller is configured to use a second convolutional neural network (CNN) to determine whether the subject is experiencing or is expected to experience an adverse medical condition.

8. The wireless medical device network of claim 1, further comprising an external gateway configured to use the ultrasonic waves to provide transcutaneous ultrasonic energy transfer to the one or more implantable devices.

9. The wireless medical device network of claim 8, wherein each of the one or more implantable devices comprises a dual-mode transducer that operates at a first frequency for signal transmissions with the wearable controller and a second frequency for signal transmissions with the external gateway.

10. The wireless medical device network of claim 9, wherein the external gateway is further configured to program or re-program the one or more implantable devices via communications over the second frequency.

11. The wireless medical device network of claim 1, wherein the adverse medical condition comprises an epileptic seizure.

12. The wireless medical device network of claim 1, wherein the system data is transmitted between the one or more implantable devices and the wearable controller in an unencrypted format.

13. A neurostimulation medical device network comprising:
one or more implantable devices for intracranial use within a subject, each of the one or more implantable devices comprising at least one stimulation means, one or more circuits for collecting system data, a receiver, and a transmitter for communications using ultrasonic waves:
a wearable controller external to the subject, the wearable controller configured to:
communicate with the one or more implantable devices using the ultrasonic waves and obtain the system data:
analyze the system data to determine whether the subject is experiencing or is expected to experience an adverse medical condition; and
communicate with the one or more implantable devices using the ultrasonic waves to apply a stimulation to treat the adverse medical condition.

14. The neurostimulation medical device network of claim 13, wherein the adverse medical condition is Parkinson's Disease.

15. The neurostimulation medical device network of claim 14, wherein:
the wearable controller comprises an accelerometer/gyroscope (AG) that collects tremor data and is further configured to:
determine whether the tremor data is indicative of Parkinson's tremors or normal body function; and
responsive to a determination that the tremor data is indicative of Parkinson's tremors, cause the one or more implantable devices to apply a stimulation to the subject.

16. The neurostimulation medical device network of claim 13, wherein wearable controller is configured to use a convolutional neural network (CNN) to analyze the system data.

17. The neurostimulation medical device network of claim 13, wherein the one or more circuits comprises a field-programmable gate array (FPGA) and microcontroller unit (MCU).

18. The neurostimulation medical device network of claim 13, wherein the one or more circuits comprises an application-specific integrated circuit (ASIC).

19. The neurostimulation medical device network of claim 13, wherein:
the one or more circuits are configured to:
continuously collect system data:
determine, using a first convolutional neural network (CNN) that the system data comprises an abnormal pattern; and
in response to the abnormal pattern being detected, initiating transmission of the system data to the wearable controller; and
the wearable controller is configured to use a second convolutional neural network (CNN) to determine whether the subject is experiencing or is expected to experience an adverse medical condition.

20. The neurostimulation medical device network of claim 13, further comprising an external gateway configured to use the ultrasonic waves to provide transcutaneous ultrasonic energy transfer to the one or more implantable devices.

21. The neurostimulation medical device network of claim 20, wherein each of the one or more implantable devices comprises a dual-mode transducer that operates at a first frequency for signal transmissions with the wearable controller and a second frequency for signal transmissions with the external gateway.

22. The neurostimulation medical device network of claim 21, wherein the external gateway is further configured to program or re-program the one or more implantable devices via communications over the second frequency.

23. The neurostimulation medical device network of claim 13, wherein the adverse medical condition comprises an epileptic seizure.

24. The neurostimulation medical device network of claim 13, wherein the system data is transmitted between the one or more implantable devices and the wearable controller in an unencrypted format.
